(19) United States
(12) Patent Application Publication
(10) Pub. No.: US 2020/0332311 A1
(43) Pub. Date: Oct. 22, 2020

(54) INCREASING PLANT BIOPRODUCT YIELD

(71) Applicant: The Texas A&M University System, College Station, TX (US)

(72) Inventors: Shuhua Yuan, College Station, TX (US); Cheng Zhao, College Station, TX (US)

(21) Appl. No.: 16/960,717

(22) PCT Filed: Jan. 12, 2018
(86) PCT No.: PCT/US2018/013571

§ 371 Date: Jul. 8, 2020

(51) Int. Cl. C12N 15/82; A01H 5/12; C12P 5/02

(52) U.S. Cl. CPC C12N 15/8269; C12Y 401/99012; C12P 5/026; A01H 5/12

(57) ABSTRACT

The present invention relates to increasing bioproduct yield in plants. In particular, the invention relates increasing the yield of bioproduct synthesized by a plant per unit mass of plant biomass. The bioproduct can be a carbon-based bioproduct, specifically it may be a terpene, and more specifically it may be squalene.

The present invention relates to increasing bioproduct yield in plants. In particular, the invention relates increasing the yield of bioproduct synthesized by a plant per unit mass of plant biomass. The bioproduct can be a carbon-based bioproduct, specifically it may be a terpene or terpenoid, and more specifically it may be squalene.

### Background

Terpenes and terpenoids are large and diverse classes of natural products. They are synthesized by plants and have broad applications as fuels, chemicals, specialty materials, nutraceuticals, and pharmaceuticals. For example, squalene is a triterpene broadly used in cosmetic, nutraceutical and pharmaceutical industries.

Despite progress in engineering terpene and terpenoid biosynthesis, several inherent challenges have limited the further increase of bioproduct yield.

Firstly, many terpene and terpenoid compounds cannot accumulate to high levels due to the existence of downstream pathways. For example, squalene production in plants, bacteria and yeast is often hampered due to downstream modification by enzymes such as hopene cyclase and squalene epoxidases.

Secondly, terpene and terpenoid biosynthesis is subject to extensive regulation, where the accumulation of end product and intermediates often lead to feedback inhibition to inactivate the key enzymes, down-regulate the pathway gene expression, and even impact the cell growth and physiology.

Thirdly, the accumulation of certain terpene compounds can be toxic to cells. As a result, plants have evolved mechanisms to address these challenges by storing terpene compounds in special plant structures such as glandular trichomes and vascular tissues. However, even though the compartmentalization of the squalene biosynthetic pathway in plastid could decrease the downstream consumption to a certain degree, terpenes such as squalene could still ‘leak’ out of the permeable chloroplast membrane according to the Fick's law and Overton Rule, and be consumed by the downstream pathways.

Previous attempts to enhance terpene production by over-expressing farnesyl pyrophosphate synthase (FPS) and squalene synthase (SQS) in the plastids in trichome led to mosaic and dwarf phenotype (reported by Wu, S., et al (2012) Engineering triterpene metabolism in tobacco, Planta 236, 867-877).

Many efforts have previously been made to increase the accumulation of squalene by reducing downstream consumption. One approach has been to inhibit squalene epoxidase. However, research suggests that squalene epoxidase is required for plant development (see, for example, Rasbery, J. M., et al (2007) Arabidopsis thaliana squalene epoxidase 1 is essential for root and seed development, Journal of Biological Chemistry 282, 17002-17013). Plants with mutations in the genes that encode a squalene epoxidase only have trace levels of squalene accumulation and exhibit other defective phenotypes.

It has also been proposed to accumulate squalene in cyanobacterium by the inactivation of hopene cyclase, the enzyme that converts squalene into hopene. Though the method deals with the reduction of downstream consumption, it is silent on how to increase terpene yield by increasing biosynthesis (see Englund, E., et al (2014) Production of squalene in Synechocystis sp. PCC 6803, PLoS One 9, e90270).

A method to enhance squalene accumulation in green algae Chlamydomonas reinhardtii has also been disclosed (see Kajikawa, M., et al (2015) Accumulation of squalene in a microalga Chlamydomonas reinhardtii by genetic modification of squalene synthase and squalene epoxidase genes, PloS one 10, e0120446). When overexpressing squalene synthase in the cytosol, squalene did not accumulate. Although partial knockdown of squalene epoxidase (SQE) leads to accumulation of squalene, co-transformation lines of SQS overexpression and SQE knockdown did not have significant difference in squalene yield, compared with SQE knockdown lines. The method indicates the difficulty of further improvement of squalene yield by overexpressing terpene synthase in a downstream suppressed system.

Various issues associated with attempts to accumulate squalene in plant cells therefore need to be addressed.

### Summary

According to a first aspect of the invention, a genetic construct is provided, comprising a promoter and a coding sequence encoding one or more peptides, wherein expression of the one or more peptides leads to an increased yield of a biological product by:

(1) reducing consumption of the bioproduct by reducing the activity of an enzyme that consumes the bioproduct; and/or (2) channeling carbon directly from photosynthesis to the production of 1-deoxy-D-xylulose 5-phosphate (DXP); and/or (3) increasing carbon fixation by photosynthesis.

In some embodiments, the bioproduct is a carbon-based bioproduct. The bioproduct may be one or more terpenes. The bioproduct may be squalene.

In some embodiments, the consumption of the bioproduct is reduced by reducing the activity of squalene epoxidase.

In some embodiments, the construct encodes artificial microRNA which mediates squalene epoxidase knockdown. The artificial microRNA may be amiRNA¹⁵⁹-SQE.

In some embodiments, the coding sequence encodes one or more further peptides, wherein expression of the one or more further peptides leads to an increased yield of the biological product by increasing the activity of squalene synthase (SQS) and/or farnesyl pyrophosphate synthase (FPS).

In some embodiments, the construct includes copies of the SQS or FPS encoding genes.

In some embodiments, the peptides cause overexpression of the SQS or FPS encoding genes.

In some embodiments, the coding sequence encodes one or more further peptides, wherein expression of the one or more further peptides leads to an increased yield of the biological product by signaling the transport of the bioproduct.

In some embodiments, the further peptide comprises a chloroplast transit peptide.

In some embodiments, carbon is channeled directly from photosynthesis to the production of 1-deoxy-D-xylulose 5-phosphate (DXP) by peptides that convert ribose-5-phosphate (R5P) or xylulose 5-phosphate (X5P) to DXP.

In some embodiments, the genetic construct encodes a mutant RibB enzyme which converts R5P or X5P to DXP. The genetic construct may encode RibB (G108S).

In some embodiments, carbon fixation by photosynthesis is increased by peptides that increase activity of the enzyme sedoheptulose-1,7-bisphosphatase (SBPase). The construct may encode SBPase.

According to a second aspect of the present invention, a recombinant vector is provided comprising the genetic construct according to the first aspect.

According to a third aspect of the present invention, a method is provided of increasing the yield of a biological product in a plant compared to the yield of the biological product in a wild-type plant cultured under the same conditions, the method comprising transforming a plant cell with the genetic construct of any one of claims 1 to 17, or the vector of claim 18, and regenerating a plant from the transformed cell.

According to a fourth aspect of the present invention, a method is provided of producing a transgenic plant which produces a yield of a biological product which is higher than that of a corresponding wild-type plant cultured under the same conditions, the method comprising transforming a plant cell with the genetic construct according to the first aspect or the vector according to the second aspect, and regenerating a plant from the transformed cell.

In some embodiments, the plant is a monocotyledonous plant. The monocotyledonous plant may be selected from the group consisting of Oryza, Arundo, Hordeum, and Triticum. Alternatively, the plant may be a dicotyledonous plant. The dicotyledonous plant may be selected from the group consisting of Arabidopsis, Nicotiana, Lycopersicon, Glycine, Brassica, Vitis, Solanum, Manihot, Arachis, Malus, Citrus, Gossypium, Lactuca, and Raphanus.

According to a fifth aspect of the present invention, a transgenic plant is provided comprising the genetic construct according to the first aspect or the vector according to the second aspect.

According to a sixth aspect of the present invention, a host cell is provided comprising the genetic construct according to the first aspect or the vector according to the second aspect.

According to a seventh aspect of the present invention, a plant propagation product is provided, obtainable from the transgenic plant of the fifth aspect.

According to an eighth aspect of the present invention, a biological product is provided, obtained from a modified plant comprising the genetic construct according to the first aspect or the vector according to the second aspect.

In some embodiments, the biological product is a terpene. The biological product may be squalene.

According to a ninth aspect of the present invention, plant part is provided containing higher levels of a biological product than a corresponding part of a wild-type plant cultured under the same conditions, wherein the plant part is harvested from the transgenic plant according to the fifth aspect or produced by the method according to the fourth aspect.

In some embodiments, the plant part is the leaf.

### BRIEF DESCRIPTION OF THE FIGURES

In order that aspects of the invention may be more fully understood, embodiments thereof are described, by way of illustrative example, with reference to the accompanying drawing in which:

FIG. 1 shows a summary of squalene biosynthesis in plants.

FIG. 2 shows putative squalene epoxidases. These are the mRNA sequences of squalene epoxidase in a phylogenetic analysis, showing that they are all similar to one another.

FIG. 3 shows the results of comparing the squalene epoxidase amino acid sequences of SEQ ID NOS: 11 to 20, encoded by the nucleic acid sequences of mRNA sequences of SEQ ID NOS: 1 to 10. The multiple regions of sequence alignment highlight the similar sequences among these genes.

FIG. 4 shows the PCR gel indicating the expression and activity of the various squalene epoxidase sequences as determined using reverse-transcriptional polymerase chain reactions.

FIGS. 5a to 5d show the sequence designs of artificial microRNA 159 (amiRNA¹⁵⁹). Underlined sequences are the target sequences of squalene epoxidase. FIGS. 5a and 5b show two sites of SQE3 only, whilst FIGS. 5c and 5d show two consensus sites of SQE3, SQE1 AND SQE2 that are targeted by artificial microRNA designs.

FIG. 6 shows constructs used to assess the effects of squalene epoxidase (SQE) suppression, overexpression of squalene synthase (SQS) and a combination thereof.

FIG. 7 shows the squalene yield in plants with the constructs shown in FIG. 6.

FIG. 8 shows a modified pathway in which the Calvin cycle has been modified by the introduction of a mutant 3,4-dihydroxy-2-butanone 4-phosphate synthase (RibB) enzyme.

FIG. 9 shows an FS-RibB construct in which the FPS and SQS are over-expressed driven by a constitutive promoter. Both enzymes are fused with a chloroplast signal peptide. In addition, a RibB enzyme is over-expressed and fused with a chloroplast signal peptide.

FIG. 10 shows the squalene content in tested plants including the FS-RibB construct io shown in FIG. 9.

FIG. 11 shows a modified pathway designed to provide an alternative route for DXP production.

FIG. 12 shows a modified pathway designed to integrate the acceleration of photosynthesis acceleration by SBPase overexpression and the C2 redirection to terpene synthesis.

FIG. 13 shows the observed increase in squalene yield (highest squalene yield from each design as shown in the left hand graph) and the increase of photosynthesis (shown in the right-hand graph).

FIG. 14 shows a pT8 plasmid map.

### DETAILED DESCRIPTION

The first principle of this invention is to reduce bioproduct consumption. In some embodiments, this is achieved by reducing squalene consumption. This will address the aforementioned issue of squalene leakage and downstream enzyme consumption which has failed to be addressed in prior art. In this invention, the activity of a squalene-consuming enzyme is suppressed in order to reduce squalene consumption and increase squalene accumulation.

In plants, squalene is converted to 2,3 squalene oxide by squalene epoxidase (SQE). In cyanobacteria and other prokaryotes squalene in converted to hopene by squalene-hopene cyclase (SHC). Thus, in some embodiments, the activity of either of these enzymes is suppressed in order to reduce squalene consumption.

In addition, activity of one or more key enzymes in the pathway for squalene synthesis is enhanced. Experiments have demonstrated a higher yield of squalene in plants with both types of modification compared with solely increasing the activity of enzymes that are involved in squalene synthesis. This may lead not only to greater squalene yield but also to greater yields of compounds derived from squalene, or greater yields of compounds from which squalene is derived.

The second principle of this invention is to directly convert 5-carbon components of the Calvin cycle, ribose-5-phosphate (R5P) and xylulose 5-phosphate (X5P), which are generated within plants during photosynthesis, to the 5-carbon 1-deoxy-D-xylulose 5-phosphate (DXP). DXP may be utilised in the synthesis of terpenes such as squalene via the non-mevalonate pathway.

The third principle is to increase the maximum rate of carbon assimilation as well as photosynthesis by removing the rate limiting step of RuBisCo reformation. This may be achieved by causing overexpression of SBPase in plants. This in turn increases the production of substrates which are utilised in the second principle to increase terpene synthesis, and thus increase the yield of terpenes, including squalene.

In this invention, the genetic modification of plants to implement one or more of the aforementioned principles is proposed. This has been found to be an effective means of increasing the terpene yield, such as squalene, in the plant without increasing the plant biomass.

Two or more of the principles described herein may be combined.

In some embodiments, the increased bioproduct synthesis is in a plant, for example a monocotyledonous plant such as one selected from the group consisting of Oryza, Arundo, Hordeum, and Triticum. Alternatively, the plant may be a dicotyledonous plant, such as one selected from the group consisting of Arabidopsis, Nicotiana, Lycopersicon, Glycine, Brassica, Vitis, Solanum, Manihot, Arachis, Malus, Citrus, Gossypium, Lactuca, and Raphanus. In some embodiments, the plant is of the genus Nicotiana, such as the species Nicotiana tabacum. In other embodiments, the plant may be algae, such as microalgae. The plant is modified to enhance bioproduct yield, such as the yield of terpenes, for example squalene, using one or more of the mechanisms described herein.

Reducing Squalene Consumption

The consumption of squalene may be reduced by reducing the activity of enzymes that have squalene as a substrate. Squalene is an intermediate in the synthesis of sterols in plants and animals, and in the synthesis of hopenoids in some bacteria. Therefore, reducing squalene consumption can lead to an increased yield of squalene.

Squalene Epoxidase Knockdown

Squalene epoxidase (SQE) (also called squalene monooxygenase) is an enzyme that uses NADPH and molecular oxygen to oxidize squalene to 2,3-oxidosqualene (squalene epoxide) in plants and animals.

According to one aspect of this invention, squalene epoxidase (SQE) activity is reduced whilst FPS and/or SQS activity is increased.

Contrary to what is suggested by the prior art, a higher yield of squalene is observed where SQE knockdown is combined with FPS and SQS overexpression, compared with FPS and SQS overexpression lines. Indeed, the experimental data demonstrates a synergistic effect, the combination of the upstream enhanced synthesis of squalene and the suppressed downstream consumption of squalene resulting in a significant increase in yield that was unexpected in view of the failure in the prior art to increase squalene yields.

In some embodiments, SQE activity may be reduced by reducing or preventing expression of the SQE genes or otherwise modifying activity of the enzyme.

In some embodiments, suppression of SQE may be achieved by preventing transcription or translation of the gene encoding SQE.

In some embodiments, SQE is suppressed by artificial microRNA mediated knockdown. This involves identifying a gene encoding squalene epoxidase and designing an artificial microRNA that complements at least part of the sequence of the SQE mRNA, to silence the RNA and prevent translation of the SQE mRNA.

The artificial microRNA is introduced into the organism to be modified to enhance squalene production, for example a plant such as a tobacco plant. This artificial microRNA knocks out the SQE, reducing the SQE activity within the cells and reducing squalene oxygenation and further conversion into sterols.

In some embodiments, squalene consumption by squalene hopene cyclase (SHC) is reduced by reducing SHC activity. This may be achieved by reducing or preventing expression of the SHC genes or otherwise modifying activity of the enzyme.

In some embodiments, suppression of SHC may be achieved by preventing transcription or translation of the gene encoding SHC.

In some embodiments, SHC is suppressed by artificial microRNA mediated knockdown. This involves identifying a gene encoding SHC and designing an artificial microRNA that complements at least part of the sequence of the SHC mRNA, to silence the RNA and prevent translation of the SHC mRNA.

Increasing Activity of Farnesyl Pyrophosphate Synthase and/or Squalene Synthase

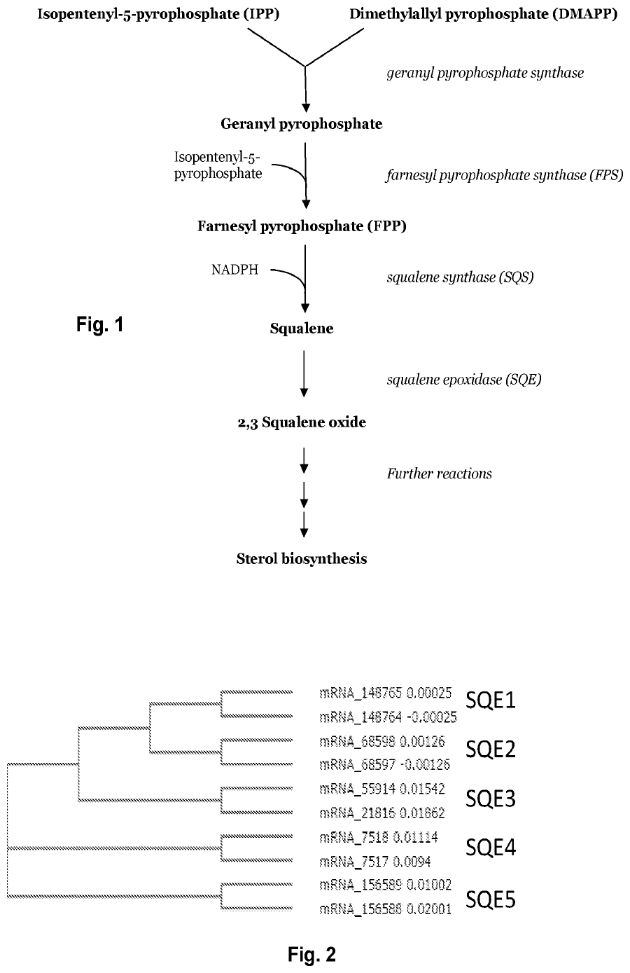

In addition to suppression of squalene consuming enzymes such as SQE and SHC, the organism may be further modified to enhance the synthesis of squalene. In some embodiments, this enhanced squalene synthesis is achieved by increasing the activity of key enzymes farnesyl pyrophosphate synthase (FPS) and/or squalene synthase (SQS). FIG. 1 shows how these enzymes are involved in squalene synthesis.

Farnesyl pyrophosphate synthase (FPPS) (also known as dimethylallyltranstransferase (DMATT) or farnesyl diphosphate synthase (FDPS)), is an enzyme that catalyses the transformation of dimethylallylpyrophosphate (DMAPP) and isopentenyl pyrophosphate (IPP) into farnesyl pyrophosphate (FPP). Geranylpyrophosphate is created in an intermediate step.

Squalene synthase (SQS) (also referred to as farnesyl-diphosphate:farnesyl-diphosphate farnesyl transferase) is an enzyme localized to the membrane of the endoplasmic reticulum. SQS catalyses a two-step reaction in which two identical molecules of farnesyl pyrophosphate (FPP) are converted into squalene via an intermediate, presqualene pyrophosphate (PSPP), with the consumption of NADPH.

SQS regulation occurs primarily at the level of SQS gene transcription. The sterol regulatory element binding protein (SREBP) class of transcription factors is important for controlling levels of SQS transcription. When sterol levels are low, an inactive form of SREBP is cleaved to form the active transcription factor, which moves to the nucleus to induce transcription of the SQS gene. Aside from SREBPs, accessory transcription factors are needed for maximal activation of the SQS promoter. Promoter studies using luciferase reporter gene assays revealed that the Sp1, and NF-Y and/or CREB transcription factors are also important for SQS promoter activation.

In squalene biosynthesis, intermediates isopentenyl diphosphate (IPP) and dimethylallyl diphosphate (DMAPP) inhibit the first enzyme of the 2-C-methyl-d-erythritol-4-phosphate (MEP) pathway for upstream terpene biosynthesis, 1-deoxyxylulose 5-phosphate synthase (DXPS). In addition, intermediate farnesyl pyrophosphate (FPP) also inhibits upstream pathway components as indicated by previous research. The farnesyl pyrophosphate synthase (FPPS), squalene synthase (SQS) or a combination thereof, for example in a protein complex, will effectively remove the pathway intermediate to enable increased squalene production. Such synergy is important for both improving the enzyme product yield and removal of pathway inhibition. The synergy comes from two effects. Firstly, the product from a first enzyme can be made immediately available to a second enzyme in an enzymatic pathway (so-called substrate channeling). The effect is the increased local concentration of the substrate for the second enzyme, thereby increasing the rate of the catalytic reaction. Secondly, the efficient utilization of the product from the first enzyme also removes the inhibitory effects of the product from the first enzyme for the entire pathway, which further improves the production.

FPS and/or SQS activity may be increased by overexpression of the FPS and/or SQS genes.

In some embodiments, activity of FPS, SQS or a combination of both is increased by inserting additional copy or copies of their genes into the organism.

In other embodiments, transcription of the genes may be enhanced, for example by

According to one embodiment, a synthetic two-enzyme complex containing farnesyl pyrophosphate synthase (FPPS) and squalene synthase (SQS) was constructed both in vitro and in vivo. In vitro results indicate the synthetic metabolons exhibited several-fold enhancement in reaction rates compared to non-complexed enzyme mixtures and such substrate synergy strongly depends on enzyme loading, substrate concentration and even ionic strength.

Compartmentalisation of Squalene

In order to ensure that the increase in the amount of squalene in the cell does not cause the abovementioned toxicity or feedback, in some embodiments, the squalene is targeted to a compartment within the cell, for example to a plastid such as the chloroplast. This also separates the squalene from the squalene consuming enzymes in the cytosol, allowing a build up in the level of the squalene.

In some embodiments, the microRNA to knock out a squalene consuming enzyme and/or the copies of genes encoding SQS and/or FPS is incorporated into the organism tagged with chloroplast transit peptides, to ensure that the products are transported to the chloroplast once expressed.

In some embodiments, the squalene may be localised in a specific compartment within the organism, for example the chloroplast, by co-expression of a compartmenting peptide, as discussed above.

Constructs and vectors may also include a transit peptide coding sequence that expresses a linked peptide that is useful for targeting of a protein product, particularly to a chloroplast. For descriptions of the use of chloroplast transit peptides, see U.S. Pat. Nos. 5,188,642 and 5,728,925. Many chloroplast-localized proteins are expressed from nuclear genes as precursors and are targeted to the chloroplast by a chloroplast transit peptide (CTP). Examples of such isolated chloroplast proteins include, but are not limited to, those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS), and transit peptides described in U.S. Pat. No. 7,193,133. It has been demonstrated in vivo and in vitro that non-chloroplast proteins may be targeted to the chloroplast by use of protein fusions with a heterologous CTP and that the CTP is sufficient to target a protein to the chloroplast. Incorporation of a suitable chloroplast transit peptide such as the Arabidopsis thaliana EPSPS CTP (CTP2) (see, Klee et al., Mol. Gen. Genet. 210:437-442, 1987) or the Petunia hybrida EPSPS CTP (CTP4) (see, della-Cioppa et al., Proc. Natl. Acad. Sci. USA 83:6873-6877, 1986) has been show to target heterologous EPSPS protein sequences to chloroplasts in transgenic plants (see, U.S. Pat. Nos. 5,627,061; 5,633,435; and 5,312,910; and EP 0218571; EP 189707; EP 508909; and EP 924299).

Experimental

5 pairs of genes in N. tabacum genome were predicted to be putative squalene epoxidase by blast using A. thaliana squalene epoxidases as templates in the N. tabacum TN90 Sierro 2014 database.

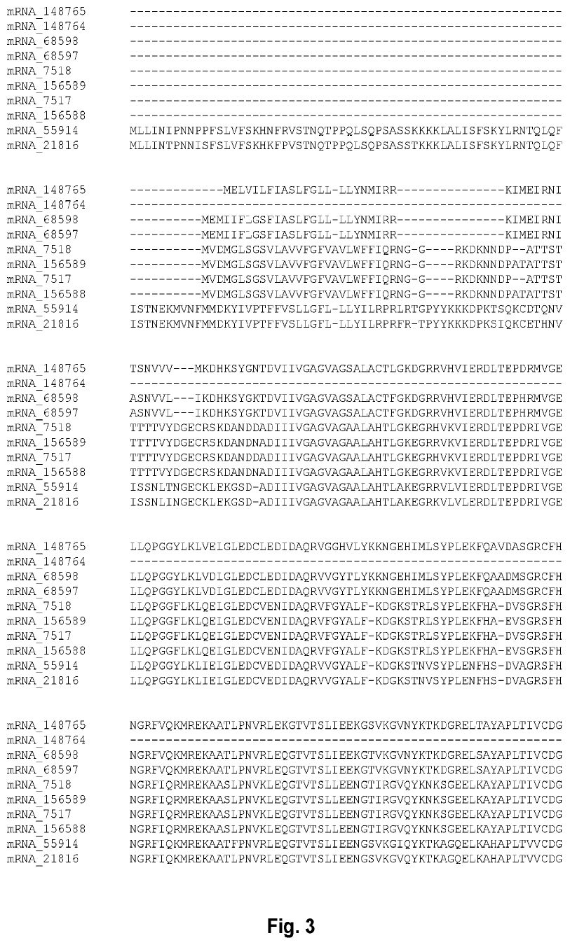

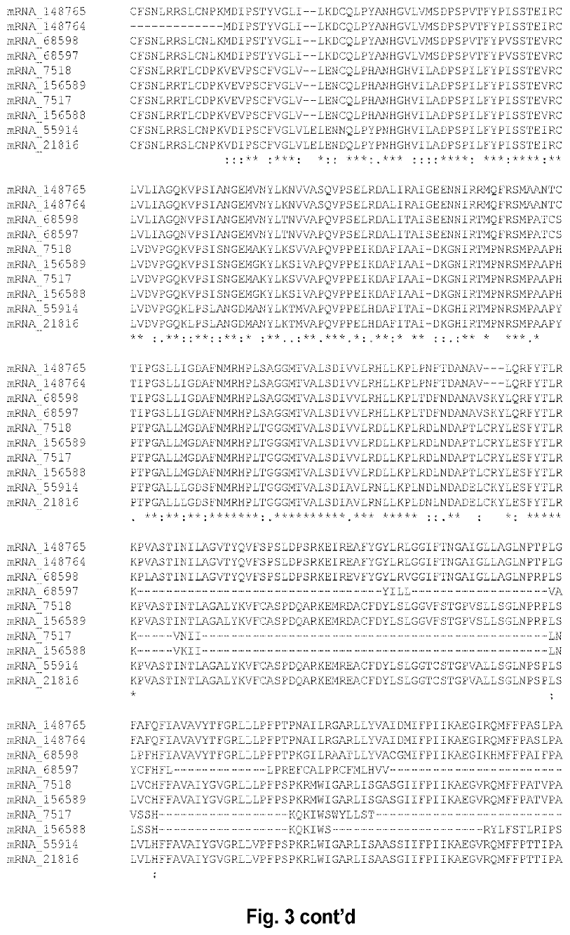

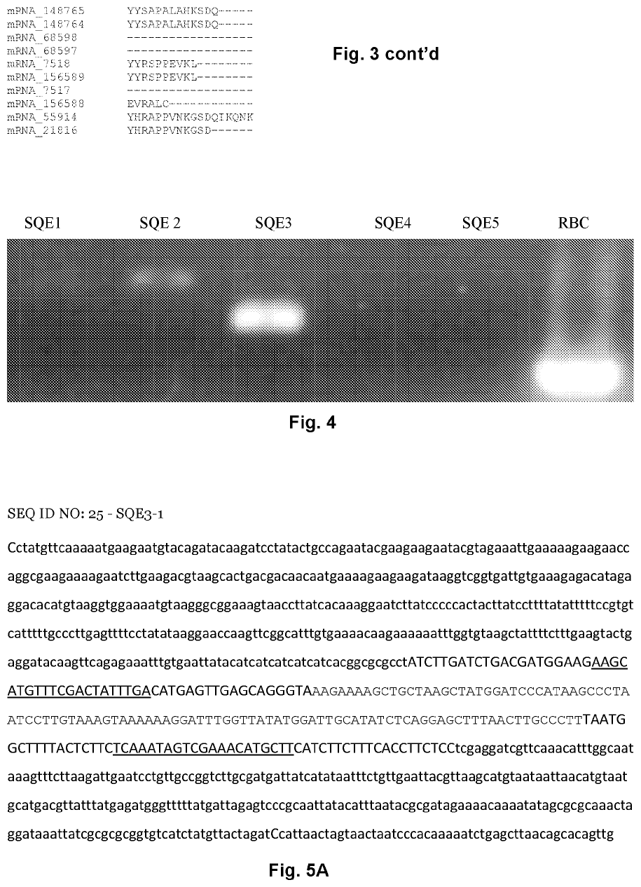

The amino acid sequences are highly similar to SQEs in other organisms. Therefore, these genes are designated as NtSQEs. Phylogenetic trees were generated using 5 pairs of the genes, as shown in FIG. 2. mRNA nucleic acid sequences and amino acid sequences are provided as SEQ ID NOS: 1 to 10 and 11 to 20, respectively and a comparison of the amino acid sequences is shown in FIG. 3.

FIG. 4 shows that SQE1 and SQE3 are the most actively expressed squalene epoxidases in tobacco leaf, as verified by reverse-transcriptional polymerase chain reactions. SQE1 and SQE2 are also expressed in leaves. Therefore, SQE3, SQE1 and SQE2 were chosen as the target genes.

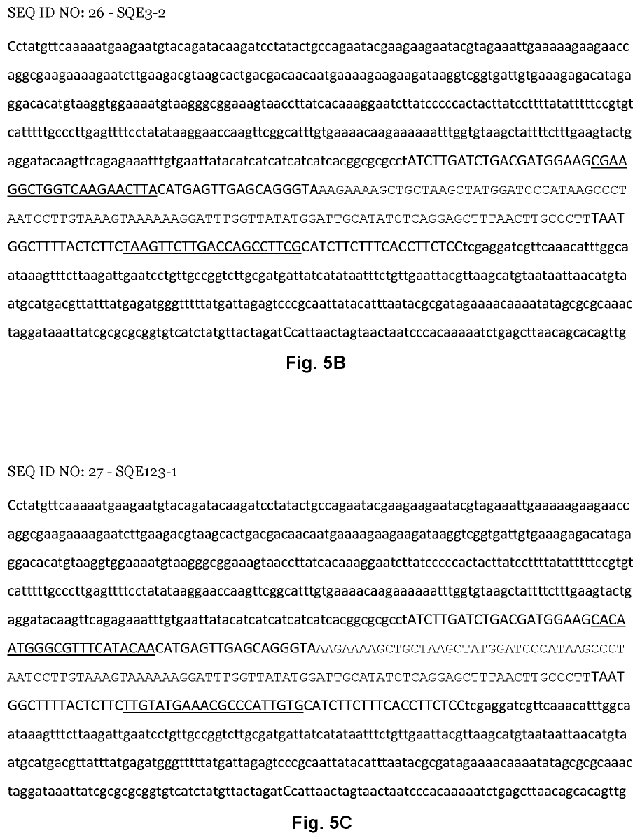

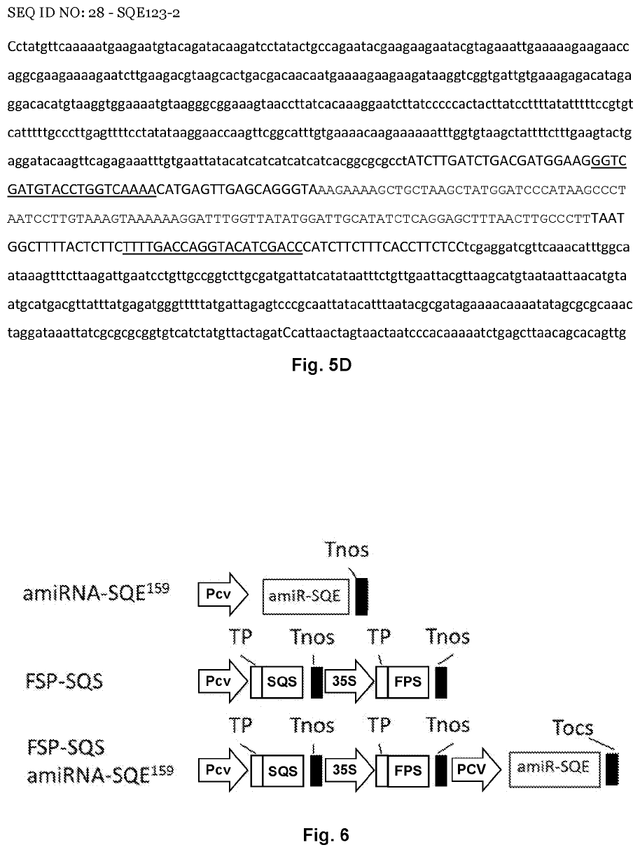

FIGS. 5a to 5d show the sequence design of amiRNA¹⁵⁹. Underlined sequences are the target sequences of squalene epoxidase. Two sites of SQE3 only and two consensus sites of SQE3, SQE1 AND SQE2 are targeted by artificial microRNA designs.

To suppress the activity of NtSQEs, the present inventors designed an artificial microRNA mediated NtSQE knockdown. A. thaliana artificial microRNA(amiRNA) 159 was used as a frame containing 21 bps sequence complemented with NtSQEs mRNA, which targets the squalene epoxidase. The amiRNA¹⁵⁹-SQE was further incorporated into commercial binary expression vector pCAMBIA 2300. The amiRNA¹⁵⁹-SQE was introduced into tobacco, together with farnesyl pyrophosphate synthase (FPS) and squalene synthase (SQS), tagged with chloroplast transit peptides (see the constructs of FIG. 6). At least to independent transformation lines were generated for FPS-SQS- amiRNA¹⁵⁹-SQE, as well as FPS-SQS and amiRNA¹⁵⁹-SQE, confirmed by genomic PCR and reverse-transcript PCR.

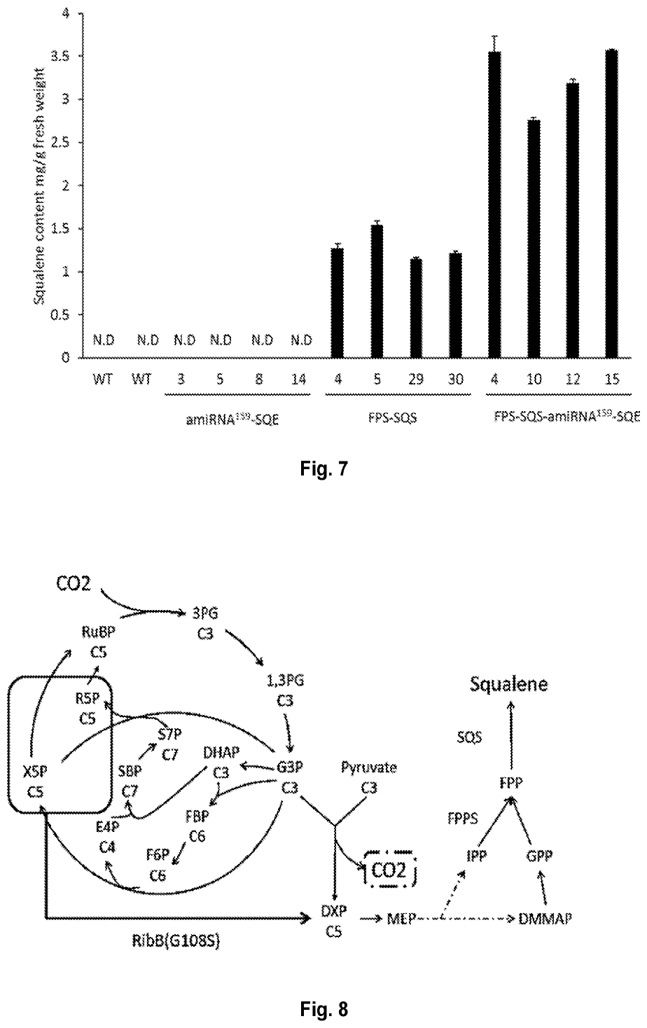

Squalene content of the tobacco leaves was measured by gas chromatography-mass spectrometry. As shown in FIG. 7, squalene content in wildtype and SQEs knock down lines are in trace level. Comparing with FPS and SQS overexpression lines, squalene content in FPS-SQS-amiRNA¹⁵⁹-SQE lines are about 3 folds higher to achieve 3.5 mg/g fresh weight. The results demonstrated that squalene yield is significantly enhanced by synergizing plastidic squalene biosynthesis with cytosol squalene epoxidases knockdown.

Enhancing the Production of Isoprenoid Precursors

The methylerythritol 4-phosphate (MEP) pathway is the source of isoprenoid precursors isopentenyl diphosphate (IDP) and dimethylallyl diphosphate (DMADP) in the plastids of plant cells.

The first reaction in the MEP pathway is two C3 molecules, pyruvate (Pyr) and glyceraldehyde 3-phosphate (G3P) are converted into 1-deoxy-D-xylulose 5-phosphate (DXP) and CO₂ by the enzyme 1-deoxy-D-xylulose-5-phosphate synthase (also known as DXP-synthase).

DXP is an intermediary component of the MEP pathway which produces two 5-carbon substrates; isopentenyl pyrophosphate (IPP) and dimethylallyl pyrophosphate (DMAPP), which are required for terpene synthesis. DXP is converted into 2-C-methyl-D-erythritol 4-phosphate (MEP) which is then broken down into IPP and DMAPP. IPP and DMAPP are terpene precursors and, as shown in FIG. 1, are substrates of FPS which produces FPP. SQS then converts FPP into squalene.

According to a further aspect of the invention, bioproduct synthesis is enhanced by providing an additional source of DXP. The bioproduct may be a terpene, such as squalene.

In some embodiments, production of DXP is increased by increasing conversion of ribose-5-phosphate (R5P) and/or xylulose 5-phosphate (X5P) to DXP. In some embodiments this is achieved by genetically modifying an organism, such as a plant, to produce an exogenous enzyme to convert R5P or X5P into DXP.

Without genetic modification these two pathways would not normally interact in this direct manner. The conversion of X5P or R5P from the Calvin cycle to DXP of the non-mevalonate pathway produces a more efficient mechanism for conversion between the 5-carbon molecules. The proposed pathway releases no CO₂ and increases carbon utilisation by a third.

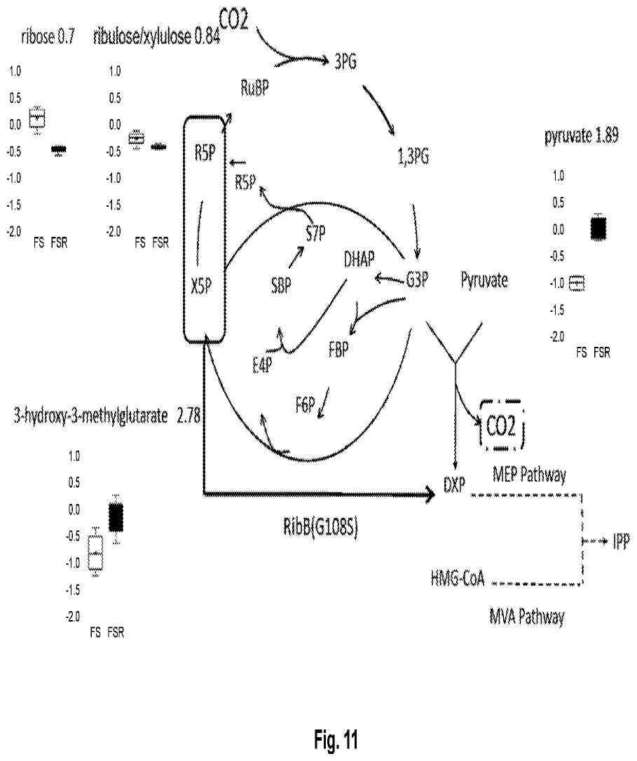

Certain mutants of 3,4-dihydroxy-2-butanone 4-phosphate synthase (RibB) catalyse the conversion of R5P to DXP. Such mutant versions of RibB may therefore be used to increase the levels of DXP in an organism, to thereby increase the production of terpenes such as squalene. FIGS. 8 and 11 show modified pathways in which the Calvin cycle has been modified by the introduction of a mutant RibB enzyme.

RibB(G108S) mutant enzyme converts R5P or xylulose 5-phosphate (X5P) to DXP. The technology has several advantages. First, it allows the direct channeling of carbon from photosynthesis (via the Calvin cycle) to terpene, enabling increased carbon flux to terpene from carbon fixation. Second, from a carbon efficiency perspective, the endogenous pathway loses one carbon out of six carbons when condensing G3P (3 carbon) and pyruvate (3 carbon) to DXP (5 carbon). The modified pathway directly channels xylulose (C5) to DXP (C5) without any carbon loss from Calvin cycle. Third, the RibB(G108S) mutant enzyme is derived from E. coli, so there will be no regulation as seen for DXP synthase (DXPS) in the MEP pathway. DXP synthase has been known as the speed-limiting enzyme subjected to extensive endogenous regulations. For example, the downstream product IPP and DMAPP can bind with DXPS to reduce its activity. RibB produces the DXPS product, DXP, but is not subject to the same endogenous regulation.

The protein sequence of a mutant RibB may be found in US 20130052692 A1 entitled Host Cells and Methods for Producing 1-Deoxyxylulose 5-phosphate (DXP) and/or a DXP Derived Compound.

The most effective mutant protein is chosen, the RibB(G108S), in which the glycine (G) is changed to serine (S) at 108^{th} ammo loci. RibB(G108S) protein sequence is provided in SEQ ID NO: 21. RibB(G108S) DNA sequence after Codon Optimization for Nicotiana tabacum (tobacco) is provided in SEQ ID NO: 22. The transit peptide (TP) sequence is provided in SEQ ID NO: 23.

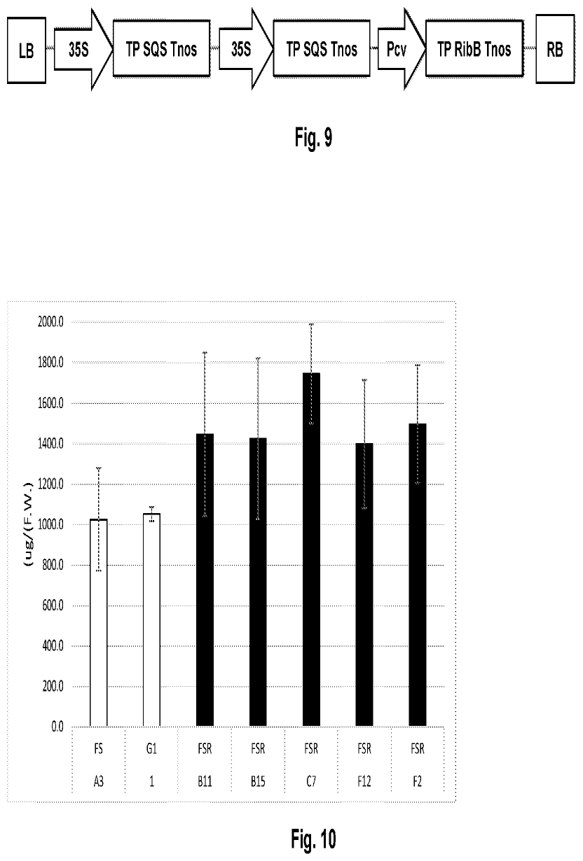

In one embodiment, an FS-RibB construct is used as shown in FIG. 9. This construct encodes not only RibB but also FFPS and SQS as, as discussed above. The FPS and SQS are over-expressed driven by a constitutive promoter. Both enzymes are fused with a chloroplast signal peptide. In addition, a RibB enzyme is over-expressed and fused with a chloroplast signal peptide. The RibB enzyme converts xylulose-5-phosphate directly into DXP, the first committed compounds in MEP pathway. The design allows the by-pass of DXPS, a heavily regulated first step enzyme of MEP, which further leads to the increase of squalene. In a further embodiment, a construct could additionally include a sequence encoding SQE.

Experimental

The RibB (G108S) mutant enzyme was optimized via codons for insertion into Nicotiana tabacum (tobacco plant). Following optimization, the genetically optimized enzyme was modified to be driven by a PCV promoter and a 210 bp TP sequence and inserted into a plasmid. The modified plasmid was designed to target the gene into the chloroplasts of Nicotiana tabacum.

Agro-bacterium mediated Nicotiana tabacum transformation was used. The GV3101 strain containing the genetically optimized FS-RibB plasmid was co-cultured on leaf dishes on Murashige and Skoog (MS) solid medium for 48 hours before being transferred onto selection medium. Following two rounds of selection, the transformers are transferred onto rooting media to generate roots before they are transferred into soil to generate T0 plants.

The To plants were grown in greenhouse conditions and further tested by Polymerase Chain Reaction (PCR) and for squalene content. Utilising the T0 seeds, five T1 plants were generated from each T0 plants to determine performance.

Squalene content was determined by collecting 0.5 g fresh leaves and grinding in liquid nitrogen. 3 ml of hexane and 90 ppm cedrene was added to the powder as an internal reference. After 2 hours of shaking, i ml of the extract was further purified by a silica column. The flow through was concentrated into 6 ml under nitrogen flow and 1 ml loaded on the GC-MS for analysis.

Five plants for each line were tested. The highest average line FSR C7 reached 1743.9 μg squalene per fresh weight, representing a 66% increase compared to the control plants (see FIG. 10).

Enhanced Photosynthesis to Support Increased Bioproduct Yield

Ribulose-1,5-bisphosphate carboxylase/oxygenase (RuBisCo) is an enzyme involved in the first major step of carbon fixation in plants and other photosynthetic organisms. The carboxylation of ribulose-1,5-bisphosphate (RuBP) by RuBisCo has been shown to be the rate limiting step in field conditions, where light typically exceeds that capable in other growing environments and atmospheric CO₂ typically is lower, especially at higher temperatures. RuBisCo binds with CO₂ and so when CO₂ concentration is low, the enzyme is limited in its capacity. Furthermore, RuBisCo side activities can lead to inhibitory products, including xylulose-1,5-bisphosphate (X5P).

Sedoheptulose-bisphosphatase (SBPase) (also known as sedoheptulose-1,7-bisphosphatase) is an enzyme that participates in the Calvin cycle and is involved in the regeneration of 5-carbon sugars in photosynthesis, including the regeneration of RuBisCo.

Overexpression of SBPase will enhance both carbon fixation and oxidation as it provides more substrate to RuBisCo, the enzyme fixing CO₂. In the present invention, an increase in the activity of SBPase will enhance photosynthesis and allow more carbon to channel to the terpene biosynthesis.

When carbon dioxide is the substrate, the product of the carboxylase reaction catalyzed by RuBisCo is a highly unstable six-carbon phosphorylated intermediate which decays almost immediately into two molecules of 3-phosphoglycerate (PGA). When oxygen is the substrate, the products of the oxygenase reaction catalyzed by RuBisCo are phosphoglycolate and PGA. Phosphoglycolate is recycled through a sequence of reactions called photorespiration. In this process, two molecules of phosphoglycolate are converted to one molecule of carbon dioxide and one molecule of PGA, which can re-enter the Calvin cycle. At normal levels of carbon dioxide and oxygen, the ratio of the reactions is about 4 to 1, which results in a net carbon dioxide fixation of only 3.5. Thus, the inability of the enzyme to prevent the reaction with oxygen greatly reduces the photosynthetic capacity of many plants.

While increased carbon fixation is adventitious for all crop production, the engineering of stable transformants utilizing SBPase in tandem with downstream bioproduct engineering and carbon partition strategies has the capability to (1) overcome carbon starvation and sugar/starch digestion to make up metabolic gap of producing bioproduct allowing for (1a) comparable or better biomass than non-SBPase strategies and/or (1b) increase carbon pool and thus flux to general metabolism allowing for increased flux into the MEP pathway. It may also (2) confer the ability to withstand abiotic stress better than non-SBPase engineered lines and WT tobacco.

Two molecules of PGA are reduced to form one molecule of glyceraldehyde 3-phosphate (G3P), which is required to form DXP.

According to an aspect of the invention, bioproduct yield is enhanced by increasing carbon fixation. In some embodiments, the bioproduct is one or more terpenes. In some embodiments, the bioproduct is squalene.

In some embodiments, bioproduct synthesis is enhanced by increasing the activity of SBPase. In some embodiments, SBPase is over expressed to enhance the carbon fixation and oxidation, increasing the production of PGA.

FIG. 11 shows the key metabolite changes in the plants engineered with RibB (FSR) and without RibB (FS—FPS and SQS only). FIG. 11 shows that ribose, ribulose, and xylulose all decreased because of the pathway rechanneling. On the other hand, because of the alternative pathway for DXP production, pyruvate increased in the engineered plant, indicating the effectiveness of the pathway design, where ribose and ribulose were consumed for DXP directly. In addition, due to the stronger flux to the MEP pathway, the HMG-CoA for MVA pathway increased, presumably due to the feedback from the higher flux of IPP and DMAPP, downstream terpene intermediate. Considering an alternative pathway exists for DXP, less pyruvate were consumed for DXP. Interestingly, the first product for the competing MEV pathway, 3-hydroxyl-3-methylglutarate was also accumulated to a higher level, indicating the downstream products from a more effective MEP may have reduce the consumption of MEV pathway.

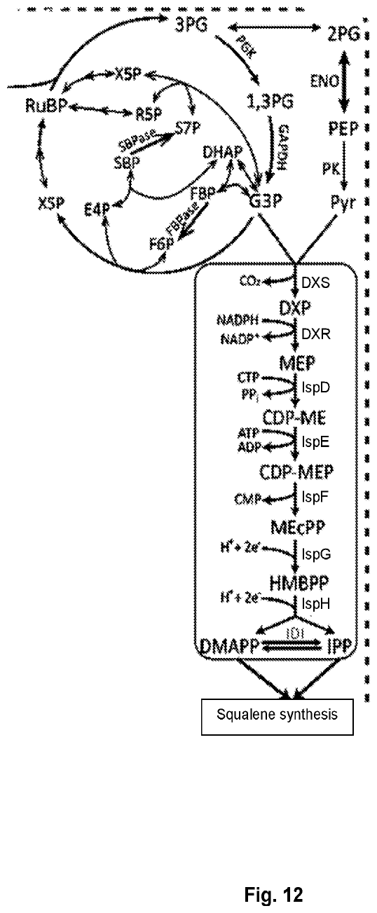

In some embodiments, a modified pathway as shown in FIG. 12 combines C2 redirection with SBPase over expression to enhance the carbon fixation and oxidation. The net results should be increased photosynthesis rate or carbon assimilation rate, and enhanced terpene synthesis.

C2 redirection was disclosed in U.S. Patent Publication No. US 2014/0283219. In the disclosed invention, a gene from a bacterial glycolate catabolic cycle was introduced into a plant to result in photorespiration bypass. Enzymes of the glycolate catabolic cycle that may be useful include glycolate dehydrogenase (GDH), glycolate oxidase (GO), malate synthase (MS), or catalase (CAT). This photorespiration and photosynthesis bypass may be coupled with downstream terpene synthesis through overexpression of, for example, FPPS, SQS, in the plant terpene synthesis pathway.

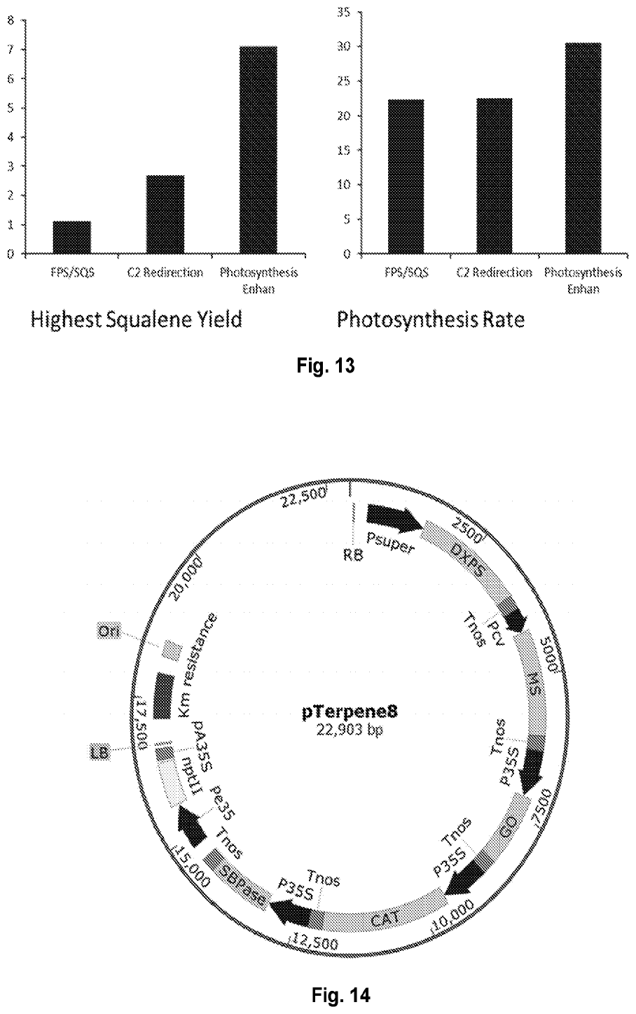

As shown in FIG. 13, the highest squalene yield observed in the SBPase+C2 redirection lines are 7.1 mg/G FW. In other words, it is almost 7% of dry weight. In addition, the photosynthesis rate increased by about 20%. The detailed design of constructs and the redesign of SBPase gene are as shown in the SEQ ID NO: 24 and in the pT8 plasmid design shown in FIG. 14.

pTerpene 8 consists of the elements in pTerpene 5 to reroute photorespiration products toward the MEP pathway utilizing constitutive expression of the photorespiration bypass along with DXPS and SBPase. The photorespiration bypass consists of glycolate oxidase, malate synthase, and catalase. DXPS is used to shunt carbon into the first committed step in the MEP pathway. SBPase is used to increase photosynthetic capacity, leading to increased carbon fixation, and supplying adequate carbon for the strong downstream carbon sink utilized for terpene synthesis. In other words, SBPase will increase both carbon fixation and carbon oxidation (photorespiration and its by-pass). In this design, both the C2 redirection (photorespiration by-pass) and carbon fixation will increase, which further increase the terpene yield.

DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of Title 37 of the United States Code of Federal Regulations § 1.822, and set forth in the tables in WIPO Standard ST.25 (1998), Appendix 2, Tables 1 and 3.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a DNA molecule, or fragment thereof, as disclosed in the present invention. For example, polymerase chain reaction (PCR) technology can be used to amplify a particular starting DNA molecule and/or to produce variants of the original molecule. DNA molecules, or fragments thereof, can also be obtained by other techniques, such as by directly synthesizing the fragment by chemical means, as is commonly practiced by using an automated oligonucleotide synthesizer.

Regulatory Elements

A regulatory element is a DNA molecule having gene regulatory activity, i.e. one that has the ability to affect the transcription and/or translation of an operably linked transcribable polynucleotide molecule. The term “gene regulatory activity” thus refers to the ability to affect the expression pattern of an operably linked transcribable polynucleotide molecule by affecting the transcription and/or translation of that operably linked transcribable polynucleotide molecule. As used herein, a transcriptional regulatory expression element group may be comprised of expression elements, such as enhancers, promoters, leaders, and introns, operably linked. Thus, a transcriptional regulatory expression element group may be comprised, for instance, of a promoter operably linked 5′ to a leader sequence, which is in turn operably linked 5′ to an intron sequence. The intron sequence may be comprised of a sequence beginning at the point of the first intron/exon splice junction of the native sequence and may be further comprised of a small leader fragment comprising the second intron/exon splice junction so as to provide for proper intron/exon processing to facilitate transcription and proper processing of the resulting transcript. Leaders and introns may positively affect transcription of an operably linked transcribable polynucleotide molecule as well as translation of the resulting transcribed RNA. The pre-processed RNA molecule comprises leaders and introns, which may affect the post-transcriptional processing of the transcribed RNA and/or the export of the transcribed RNA molecule from the cell nucleus into the cytoplasm. Following post-transcriptional processing of the transcribed RNA molecule, the leader sequence may be retained as part of the final messenger RNA and may positively affect the translation of the messenger RNA molecule.

Regulatory elements such as promoters, leaders, introns, and transcription termination regions are DNA molecules that have gene regulatory activity and play an integral part in the overall expression of genes in living cells. The term “regulatory element” refers to a DNA molecule having gene regulatory activity, i.e. one that has the ability to affect the transcription and/or translation of an operably linked transcribable polynucleotide molecule. Isolated regulatory elements, such as promoters and leaders, which function in plants are therefore useful for modifying plant phenotypes through the methods of genetic engineering.

Regulatory elements may be characterized by their expression pattern effects (qualitatively and/or quantitatively), e.g. positive or negative effects and/or constitutive or other effects, such as by their temporal, spatial, developmental, tissue, environmental, physiological, pathological, cell cycle, and/or chemically responsive expression pattern, and any combination thereof, as well as by quantitative or qualitative indications. A promoter may be useful as a regulatory element for modulating the expression of an operably linked transcribable polynucleotide molecule.

As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as an mRNA, a dsRNA, a tRNA, an rRNA, a miRNA, and the like.

As used herein, the term “protein expression” is any pattern of translation of a transcribed RNA molecule into a protein molecule. Protein expression may be characterized by its temporal, spatial, developmental, or morphological qualities, as well as by quantitative or qualitative indications.

As used herein, the term “promoter” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, i.e. a promoter produced through the fusion of two or more heterologous DNA molecules. In specific embodiments of the invention, such molecules and any variants or derivatives thereof as described herein are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” that provides a basal level of transcription and is comprised of a TATA box or equivalent sequence for recognition and binding of the RNA polymerase II complex for initiation of transcription.

Compositions derived from a promoter useful for the present invention, such as internal or 5′ deletions, for example, can be produced using methods known in the art to improve or alter expression, including by removing elements that have either positive or negative effects on expression; duplicating elements that have positive or negative effects on expression; and/or duplicating or removing elements that have tissue- or cell-specific effects on expression. Further deletions can be made to remove any elements that have positive or negative; tissue specific; cell specific; or timing specific (such as, but not limited to, circadian rhythms) effects on expression. The efficacy of the modifications, duplications or deletions described herein on the desired expression aspects of a particular transgene may be tested empirically in stable and transient plant assays, such as those described in the working examples herein, so as to validate the results, which may vary depending upon the changes made and the goal of the change in the starting molecule.

As used herein, the term “leader” refers to a DNA molecule isolated from the untranslated 5′ region (5′ UTR) of a genomic copy of a gene and defined generally as a nucleotide segment between the transcription start site (TSS) and the protein coding sequence start site. Alternately, leaders may be synthetically produced or manipulated DNA elements. A leader can be used as a 5′ regulatory element for modulating expression of an operably linked transcribable polynucleotide molecule. Leader molecules may be used with a heterologous promoter or with their native promoter. Promoter molecules of the present invention may thus be operably linked to their native leader or may be operably linked to a heterologous leader. In specific embodiments, such sequences may be provided defined as being capable of acting as a leader in a host cell, including, for example, a transgenic plant cell. In one embodiment, such sequences are decoded as comprising leader activity.

A leader sequence (5′ UTR) in accordance with the present invention may be comprised of regulatory elements or may adopt secondary structures that can have an effect on transcription or translation of a transgene. Such a leader sequence may be used in accordance with the present invention to make chimeric regulatory elements that affect transcription or translation of a transgene. In addition, such a leader sequence may be used to make chimeric leader sequences that affect transcription or translation of a transgene.

The introduction of a foreign gene into a new plant host does not always result in high expression of the incoming gene. Furthermore, if dealing with complex traits, it is sometimes necessary to modulate several genes with spatially or temporally different expression pattern. Introns can principally provide such modulation. However, multiple uses of the same intron in one plant have been shown to exhibit disadvantages. In those cases, it is necessary to have a collection of basic control elements for the construction of appropriate recombinant DNA elements. The number of introns known in the art to have expression-enhancing properties is limited, and thus, alternatives are needed.

In accordance with the present invention, a promoter or promoter fragment may be analyzed for the presence of known promoter elements, i.e. DNA sequence characteristics, such as a TATA-box and other known transcription factor binding site motifs. Identification of such known promoter elements may be used by one of skill in the art to design variants of a promoter having a similar expression pattern to the original promoter.

As used herein, the term “enhancer” or “enhancer element” refers to a cis-acting transcriptional regulatory element (a cis-element), which confers an aspect of the overall expression pattern, but is usually insufficient alone to drive transcription of an operably linked polynucleotide sequence. Unlike promoters, enhancer elements do not usually include a transcription start site (TSS), or TATA box or equivalent sequence. A promoter may naturally comprise one or more enhancer elements that affect the transcription of an operably linked polynucleotide sequence. An isolated enhancer element may also be fused to a promoter to produce a chimeric promoter cis-element, which confers an aspect of the overall modulation of gene expression. A promoter or promoter fragment may comprise one or more enhancer elements that affect the transcription of operably linked genes. Many promoter enhancer elements are believed to bind DNA-binding proteins and/or affect DNA topology, producing local conformations that selectively allow or restrict access of RNA polymerase to the DNA template, or that facilitate selective opening of the double helix at the site of transcriptional initiation. An enhancer element may function to bind transcription factors that regulate transcription. Some enhancer elements bind more than one transcription factor, and transcription factors may interact with different affinities with more than one enhancer domain. Enhancer elements can be identified by a number of techniques, including deletion analysis, i.e. deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-element motifs or enhancer elements as a target sequence or target motif with conventional DNA sequence comparison methods, such as BLAST. The fine structure of an enhancer domain can be further studied by mutagenesis (or substitution) of one or more nucleotides or by other conventional methods. Enhancer elements can be obtained by chemical synthesis or by isolation from regulatory elements that include such elements, and they can be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed by the present invention.

In plants, the inclusion of some introns in gene constructs leads to increased mRNA and protein accumulation relative to constructs lacking the intron. This effect has been termed “intron mediated enhancement” (IME) of gene expression (Mascarenhas et al., (1990) Plant Mol. Biol. 15:913-920). Introns known to stimulate expression in plants have been identified in maize genes [e.g., tubA1, Adh1, Sh1, Ubi1 (Jeon et al., Plant Physiol. 123:1005-1014, 2000; Callis et al., Genes Dev. 1:1183-1200, 1987; Vasil et al., Plant Physiol. 91:1575-1579, 1989; Christiansen et al., Plant Mol. Biol. 18:675-689, 1992) and in rice genes (e.g., salt, tpi: McElroy et al., Plant Cell 2:163-171, 1990; Xu et al., Plant Physiol. 106:459-467, 1994). Similarly, introns from dicotyledonous plant genes such as petunia (e.g., rbcS), potato (e.g., st-ls1) and Arabidopsis thaliana (e.g., ubq3 and pat1) have been found to elevate gene expression rates (Dean et al., Plant Cell 1:201-208, 1989; Leon et al., Plant Physiol. 95:968-972, 1991; Norris et al., Plant Mol Biol. 21:895-906, 1993; Rose and Last, Plant J.11:455-464, 1997). It has been shown that deletions or mutations within the splice sites of an intron reduce gene expression, indicating that splicing might be needed for IME (Mascarenhas et al., Plant Mol Biol. 15:913-920, 1990; Clancy and Hannah, Plant Physiol. 130:918-929, 2002). However, such splicing is not required for a certain IME in dicotyledonous plants, as shown by point mutations within the splice sites of the pan gene from A. thaliana (Rose and Beliakoff, Plant Physiol. 122:535-542, 2000).

Enhancement of gene expression by introns is not a general phenomenon because some intron insertions into recombinant expression cassettes fail to enhance expression (e.g., introns from dicot genes such as the rbcS gene from pea, the phaseolin gene from bean, and the stls-1 gene from Solanum tuberosum) and introns from maize genes (the ninth intron of the adh1 gene, and the first intron of the hsp81 gene) (Chee et al., Gene 41:47-57, 1986; Kuhlemeier et al., Mol Gen Genet 212:405-411, 1988; Mascarenhas et al., Plant Mol. Biol. 15:913-920, 1990; Sinibaldi and Mettler, In WE Cohn, K Moldave, eds, Progress in Nucleic Acid Research and Molecular Biology, Vol 42. Academic Press, New York, pp 229-257, 1992; Vancanneyt et al., Mol. Gen. Genet. 220:245-250, 1990). Therefore, not every intron can be employed to manipulate the gene expression level of non-endogenous genes or endogenous genes in transgenic plants. What characteristics or specific sequence features must be present in an intron sequence in order to enhance the expression rate of a given gene is not known in the prior art, and therefore it is not possible to predict whether a given plant intron, when used heterologously, will cause IME.

As used herein, the term “chimeric” refers to a single DNA molecule produced by fusing a first DNA molecule to a second DNA molecule, where neither the first nor second the DNA molecule would normally be found in that configuration, i.e. fused to the other.

The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments, for example the fusion of a promoter to an enhancer element. Thus, the design, construction, and use of chimeric promoters according to the methods disclosed herein for modulating the expression of operably linked transcribable polynucleotide molecules are encompassed by the present invention.

As used herein, the term “variant” refers to a second DNA molecule that is similar in composition, but not identical to, a first DNA molecule, and yet the second DNA molecule still maintains the general functionality, i.e. same or similar expression pattern, of the first DNA molecule. A variant may be a shorter or truncated version of the first DNA molecule and/or an altered version of the sequence of the first DNA molecule, such as one with different restriction enzyme sites and/or internal deletions, substitutions, and/or insertions. A “variant” may also encompass a regulatory element having a nucleotide sequence comprising a substitution, deletion, and/or insertion of one or more nucleotides of a reference sequence, wherein the derivative regulatory element has more or less or equivalent transcriptional or translational activity than the corresponding parent regulatory molecule. The regulatory element “variants” will also encompass variants arising from mutations that naturally occur in bacterial and plant cell transformation. In the present invention, a polynucleotide sequence may be used to create variants that are similar in composition, but not identical to, the polynucleotide sequence of the original regulatory element, while still maintaining the general functionality, i.e. same or similar expression pattern, of the original regulatory element. Production of such variants of the present invention is well within the ordinary skill of the art in light of the disclosure and is encompassed within the scope of the present invention. Chimeric regulatory element “variants” comprise the same constituent elements as a reference sequence, but the constituent elements comprising the chimeric regulatory element may be operatively linked by various methods known in the art, such as restriction enzyme digestion and ligation, ligation independent cloning, modular assembly of PCR products during amplification, or direct chemical synthesis of the regulatory element, as well as other methods known in the art. The resulting chimeric regulatory element “variant” can be comprised of the same, or variants of the same, constituent elements of the reference sequence but differ in the sequence or sequences that comprise the linking sequence or sequences which allow the constituent parts to be operatively linked.

Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule, where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell. A vector according to the present invention may include an expression cassette or transgene cassette isolated from any of the aforementioned molecules.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. A leader, for example, is operably linked to coding sequence when it is capable of serving as a leader for the polypeptide encoded by the coding sequence.

Constructs of the present invention may be provided, in one embodiment, as double Ti plasmid border DNA constructs that have right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells that permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes a Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present invention.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art (see, for example, Molecular Cloning: A Laboratory Manual, 3rd edition Volumes 1, 2, and 3, J. Sambrook, D. W. Russell, and N. Irwin, Cold Spring Harbor Laboratory Press, 2000). Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, 1988; and Glick et al., Methods in Plant Molecular Biology and Biotechnology, CRC Press, Boca Raton, Fla., 1993). Typical vectors useful for expression of nucleic acids in higher plants are well known in the art and include vectors derived from the tumor-inducing (Ti) plasmid of A. tumefaciens (Rogers et al., Methods in Enzymology 153: 253-277, 1987). Other recombinant vectors useful for plant transformation, including the pCaMVCN transfer control vector, have also been described in the scientific literature (see, for example, Fromm et al., Proc. Natl. Acad. Sci. USA 82: 5824-5828, 1985).

Various regulatory elements may be included in a construct including any of those provided herein. Any such regulatory elements may be provided in combination with other regulatory elements. Such combinations can be designed or modified to produce desirable regulatory features. In one embodiment, constructs of the present invention comprise at least one regulatory element operably linked to a transcribable polynucleotide molecule operably linked to a 3′ transcription termination molecule.

Constructs of the present invention may include any promoter or leader provided herein or known in the art. For example, a promoter of the present invention may be operably linked to a heterologous non-translated 5′ leader such as one derived from a heat shock protein gene (see, for example, U.S. Pat. Nos. 5,659,122 and 5,362,865). Alternatively, a leader of the present invention may be operably linked to a heterologous promoter such as the Cauliflower Mosaic Virus (CaMV) 35S transcript promoter (see, U.S. Pat. No. 5,352,605).

As used herein, the term “intron” refers to a DNA molecule that may be isolated or identified from the genomic copy of a gene and may be defined generally as a region spliced out during mRNA processing prior to translation. Alternately, an intron may be a synthetically produced or manipulated DNA element. An intron may contain enhancer elements that effect the transcription of operably linked genes. An intron may be used as a regulatory element for modulating expression of an operably linked transcribable polynucleotide molecule. A DNA construct may comprise an intron, and the intron may or may not be heterologous with respect to the transcribable polynucleotide molecule sequence. Examples of introns in the art include the rice actin intron (U.S. Pat. No. 5,641,876) and the corn HSP70 intron (U.S. Pat. No. 5,859,347). Further, when modifying intron/exon boundary sequences, it may be preferable to avoid using the nucleotide sequence AT or the nucleotide A just prior to the 5′ end of the splice site (GT) and the nucleotide G or the nucleotide sequence TG, respectively, immediately after 3′ end of the splice site (AG) to eliminate the potential of unwanted start codons formed during processing of the messenger RNA into the final transcript. The sequence around the 5′ or 3′ end splice junction sites of the intron can thus be modified in this manner.

As used herein, the term “3′ transcription termination molecule” or “3′ UTR” refers to a DNA molecule that is used during transcription to produce the 3′ untranslated region (3′ UTR) of an mRNA molecule. The 3′ untranslated region of an mRNA molecule may be generated by specific cleavage and 3′ polyadenylation (polyA tail). A 3′ UTR may be operably linked to and located downstream of a transcribable polynucleotide molecule and may include polynucleotides that provide a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region (see, Fraley, et al., Proc. Natl. Acad. Sci. USA, 80: 4803-4807, 1983); wheat hsp17 3′ region; pea rubisco small subunit 3′ region; cotton E6 3′ region (U.S. Pat. No. 6,096,950); 3′ regions disclosed in WO/0011200 A2; and the coixin 3′ UTR (U.S. Pat. No. 6,635,806).

3′ UTRs typically find beneficial use for the recombinant expression of specific genes. In animal systems, machinery of 3′ UTRs has been well defined (e.g. Zhao et al., Microbiol Mol Biol Rev 63:405-445, 1999; Proudfoot, Nature 322:562-565, 1986; Kim et al., Biotechnology Progress 19:1620-1622, 2003; Yonaha and Proudfoot, EMBO J. 19:3770-3777, 2000; Cramer et al., FEBS Letters 498:179-182, 2001; Kuerstem and Goodwin, Nature Reviews Genetics 4:626-637, 2003). Effective termination of RNA transcription is required to prevent unwanted transcription of trait-unrelated (downstream) sequences, which may interfere with trait performance. Arrangement of multiple gene expression cassettes in local proximity to one another (e.g. within one T-DNA) may cause suppression of gene expression of one or more genes in said construct in comparison to independent insertions (Padidam and Cao, BioTechniques 31:328-334, 2001. This may interfere with achieving adequate levels of expression, for instance in cases where strong gene expression from all cassettes is desired.

In plants, clearly defined polyadenylation signal sequences are not known. Hasegawa et al. (Plant J. 33:1063-1072, 2003) were not able to identify conserved polyadenylation signal sequences in both in vitro and in vivo systems in Nicotiana sylvestris and to determine the actual length of the primary (non-polyadenylated) transcript. A weak 3′ UTR may generate read-through, which may affect the expression of the genes located in neighboring expression cassettes (Padidam and Cao, BioTechniques 31:328-334, 2001). Appropriate control of transcription termination can prevent read-through into sequences (e.g. other expression cassettes) localized downstream and can further allow efficient recycling of RNA polymerase, to improve gene expression. Efficient termination of transcription (release of RNA Polymerase II from the DNA) is prerequisite for re-initiation of transcription and thereby directly affects the overall transcript level. Subsequent to transcription termination, the mature mRNA is released from the site of synthesis and template to the cytoplasm. Eukaryotic mRNAs are accumulated as poly(A) forms in vivo, making it difficult to detect transcriptional termination sites by conventional methods. However, prediction of functional and efficient 3′ UTRs by bioinformatics methods is difficult in that there are no conserved sequences to enable easy prediction of an effective 3′ UTR.

From a practical standpoint, it may be beneficial that a 3′ UTR used in a transgene cassette possesses certain characteristics. For example, a 3′ UTR useful in accordance with the present invention may efficiently and effectively terminate transcription of the transgene and prevent read-through of the transcript into any neighboring DNA sequence, which can be comprised of another transgene cassette, as in the case of multiple cassettes residing in one T-DNA, or the neighboring chromosomal DNA into which the T-DNA has inserted. The 3′ UTR optimally should not cause a reduction in the transcriptional activity imparted by the promoter, leader, and introns that are used to drive expression of the transgene. In plant biotechnology, the 3′ UTR is often used for priming of amplification reactions of reverse transcribed RNA extracted from the transformed plant and may be used to (1) assess the transcriptional activity or expression of the transgene cassette once integrated into the plant chromosome; (2) assess the copy number of insertions within the plant DNA; and (3) assess zygosity of the resulting seed after breeding. The 3′ UTR may also be used in amplification reactions of DNA extracted from the transformed plant to characterize the intactness of the inserted cassette.

3′ UTRs useful in providing expression of a transgene in plants may be identified based upon the expression of expressed sequence tags (ESTs) in cDNA libraries made from messenger RNA isolated from seed, flower, or any other tissues derived from, for example, Big bluestem (Andropogon gerardii), Plume Grass (Saccharum ravennae), Green bristlegrass (Setaria viridis), Teosinte (Zea mays subsp. mexicana), Foxtail millet (Setaria italica), or Coix (Coix lacryma-jobi). Using methods known to those skilled in the art, libraries of cDNA may be made from tissues isolated from a plant species using flower tissue, seed, leaf, root, or other plant tissues. The resulting cDNAs are sequenced using various sequencing methods known in the art. The resulting ESTs are assembled into clusters using bioinformatics software such as clc_ref_assemble_complete version 2.01.37139 (CLC bio USA, Cambridge, Mass. 02142). Transcript abundance of each cluster is determined by counting the number of cDNA reads for each cluster. The identified 3′ UTRs may be comprised of sequence derived from cDNA sequence, as well as sequence derived from genomic DNA. A cDNA sequence may be used to design primers, which may then be used with GenomeWalker™ (Clontech Laboratories, Inc, Mountain View, Calif.) libraries constructed following the manufacturer's protocol to clone the 3′ region of the corresponding genomic DNA sequence to provide a longer termination sequence. Analysis of relative transcript abundance either by direct counts or normalized counts of observed sequence reads for each tissue library may be used to infer properties about patters of expression. For example, some 3′ UTRs may be found in transcripts more abundant in root tissue rather than leaf tissue. This suggests that the transcript is highly expressed in root and that the properties of root expression may be attributable to the transcriptional regulation of the promoter, the lead, the introns or the 3′ UTR. Empirical testing of 3′ UTRs identified by the properties of expression within specific organs, tissues or cell types can result in the identification of 3′ UTRs that enhance expression in those specific organs, tissues or cell types.

Constructs and vectors may also include a transit peptide coding sequence that expresses a linked peptide that is useful for targeting of a protein product, particularly to a chloroplast, leucoplast, or other plastid organelle; mitochondria; peroxisome; vacuole; or an extracellular location. For descriptions of the use of chloroplast transit peptides, see U.S. Pat. Nos. 5,188,642 and 5,728,925. Many chloroplast-localized proteins are expressed from nuclear genes as precursors and are targeted to the chloroplast by a chloroplast transit peptide (CTP). Examples of such isolated chloroplast proteins include, but are not limited to, those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS), and transit peptides described in U.S. Pat. No. 7,193,133. It has been demonstrated in vivo and in vitro that non-chloroplast proteins may be targeted to the chloroplast by use of protein fusions with a heterologous CTP and that the CTP is sufficient to target a protein to the chloroplast. Incorporation of a suitable chloroplast transit peptide such as the Arabidopsis thaliana EPSPS CTP (CTP2) (see, Klee et al., Mol. Gen. Genet. 210:437-442, 1987) or the Petunia hybrida EPSPS CTP (CTP4) (see, della-Cioppa et al., Proc. Natl. Acad. Sci. USA 83:6873-6877, 1986) has been show to target heterologous EPSPS protein sequences to chloroplasts in transgenic plants (see, U.S. Pat. Nos. 5,627,061; 5,633,435; and 5,312,910; and EP 0218571; EP 189707; EP 508909; and EP 924299).

Transcribable Polynucleotide Molecules

As used herein, the term “transcribable polynucleotide molecule” refers to any DNA molecule capable of being transcribed into a RNA molecule, including, but not limited to, those having protein coding sequences and those producing RNA molecules having sequences useful for gene suppression. A “transgene” refers to a transcribable polynucleotide molecule heterologous to a host cell at least with respect to its location in the genome and/or a transcribable polynucleotide molecule artificially incorporated into a host cell's genome in the current or any prior generation of the cell.

A promoter of the present invention may be operably linked to a transcribable polynucleotide molecule that is heterologous with respect to the promoter molecule. As used herein, the term “heterologous” refers to the combination of two or more polynucleotide molecules when such a combination is not normally found in nature. For example, the two molecules may be derived from different species and/or the two molecules may be derived from different genes, e.g. different genes from the same species, or the same genes from different species. A promoter is thus heterologous with respect to an operably linked transcribable polynucleotide molecule if such a combination is not normally found in nature, i.e. that transcribable polynucleotide molecule is not naturally occurring operably linked in combination with that promoter molecule.

The transcribable polynucleotide molecule may generally be any DNA molecule for which expression of a RNA transcript is desired. Such expression of an RNA transcript may result in translation of the resulting mRNA molecule and thus protein expression. Alternatively, for example, a transcribable polynucleotide molecule may be designed to ultimately cause decreased expression of a specific gene or protein. In one embodiment, this may be accomplished by using a transcribable polynucleotide molecule that is oriented in the antisense direction. One of ordinary skill in the art is familiar with using such antisense technology. Briefly, as the antisense transcribable polynucleotide molecule is transcribed, the RNA product hybridizes to and sequesters a complimentary RNA molecule inside the cell. This duplex RNA molecule cannot be translated into a protein by the cell's translational machinery and is degraded in the cell. Any gene may be negatively regulated in this manner.

Thus, in one embodiment of the present invention, a regulatory element may be operably linked to a transcribable polynucleotide molecule on order to modulate transcription of the transcribable polynucleotide molecule at a desired level or in a desired pattern when the construct is integrated in the genome of a plant cell. In one embodiment, the transcribable polynucleotide molecule comprises a protein-coding region of a gene, and the promoter affects the transcription of an RNA molecule that is translated and expressed as a protein product. In another embodiment, the transcribable polynucleotide molecule comprises an antisense region of a gene, and the promoter affects the transcription of an antisense RNA molecule, double stranded RNA or other similar inhibitory RNA molecule in order to inhibit expression of a specific RNA molecule of interest in a target host cell.

Genes of Agronomic Interest

Transcribable polynucleotide molecules in accordance with the present invention may be genes of agronomic interest. As used herein, the term “gene of agronomic interest” refers to a transcribable polynucleotide molecule that, when expressed in a particular plant tissue, cell, or cell type, confers a desirable characteristic, such as one associated with plant morphology, physiology, growth, development, yield, product, nutritional profile, disease or pest resistance, and/or environmental or chemical tolerance. Genes of agronomic interest include, but are not limited to, those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent, such as an antisense or RNAi molecule targeting a particular gene for suppression. The product of a gene of agronomic interest may act within the plant in order to cause an effect upon the plant physiology or metabolism, or may be act as a pesticidal agent in the diet of a pest that feeds on the plant.

In one embodiment of the present invention, a promoter is incorporated into a construct such that the promoter is operably linked to a transcribable polynucleotide molecule that is a gene of agronomic interest. The expression of the gene of agronomic interest is desirable in order to confer an agronomically beneficial trait. Without limitation, a beneficial agronomic trait may include, for example, herbicide tolerance, insect control, modified yield, fungal disease resistance, virus resistance, nematode resistance, bacterial disease resistance, plant growth and development, starch production, modified oil production, high oil production, modified fatty acid content, high protein production, fruit ripening, enhanced animal and human nutrition, biopolymers, environmental stress resistance, pharmaceutical peptides and secretable peptides, improved processing traits, improved digestibility, enzyme production, flavor, nitrogen fixation, hybrid seed production, fiber production, and biofuel production, among others. Examples of genes of agronomic interest known in the art include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. USRE 38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658; 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat, Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; and 6,476,295), modified oil production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,541,259; 5,985,605; and 6,171,640), biopolymers (U.S. Pat. Nos. USRE 37,543; 6,228,623; 5,958,745; and 6,946,588), environmental stress resistance (U.S. Pat. No. 6,072,103), pharmaceutical peptides and secretable peptides (U.S. Pat, Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

Alternatively, a gene of agronomic interest can affect the above mentioned plant characteristic or phenotype by encoding an RNA molecule that causes the targeted modulation of gene expression of an endogenous gene, for example via antisense (see for example, U.S. Pat. No. 5,107,065); inhibitory RNA (“RNAi,” including modulation of gene expression via mechanisms mediated by miRNA, siRNA, transacting siRNA, and phased sRNA, e.g. as described in published applications US 2006/0200878 and US 2008/0066206, and in U.S. patent application Ser. No. 11/974,469); or cosuppression-mediated mechanisms. The RNA may also be a catalytic RNA molecule (e.g. a ribozyme or a riboswitch; see e.g. US 2006/0200878) engineered to cleave a desired endogenous mRNA product. Thus, any transcribable polynucleotide molecule that encodes a transcribed RNA molecule that affects an agronomically important phenotype or morphology change of interest may be useful for the practice of the present invention. Methods are known in the art for constructing and introducing constructs into a cell in such a manner that the transcribable polynucleotide molecule is transcribed into a molecule that is capable of causing gene suppression. For example, posttranscriptional gene suppression using a construct with an anti-sense oriented transcribable polynucleotide molecule to regulate gene expression in plant cells is disclosed in U.S. Pat. Nos. 5,107,065 and 5,759,829, and posttranscriptional gene suppression using a construct with a sense-oriented transcribable polynucleotide molecule to regulate gene expression in plants is disclosed in U.S. Pat. Nos. 5,283,184 and 5,231,020. Expression of a transcribable polynucleotide in a plant cell can also be used to suppress plant pests feeding on the plant cell, for example, compositions isolated from coleopteran pests (U.S. Patent Publication No. US20070124836) and compositions isolated from nematode pests (U.S. Patent Publication No. US20070250947). Plant pests include, but are not limited to arthropod pests, nematode pests, and fungal or microbial pests. Exemplary transcribable polynucleotide molecules for incorporation into constructs of the present invention include, for example, DNA molecules or genes from a species other than the target species or genes that originate with or are present in the same species, but are incorporated into recipient cells by genetic engineering methods rather than classical reproduction or breeding techniques. The type of polynucleotide molecule may include, but is not limited to, a polynucleotide molecule that is already present in the plant cell, a polynucleotide molecule from another plant, a polynucleotide molecule from a different organism, or a polynucleotide molecule generated externally, such as a polynucleotide molecule containing an antisense message of a gene, or a polynucleotide molecule encoding an artificial, synthetic, or otherwise modified version of a transgene.

Selectable Markers

As used herein the term “marker” refers to any transcribable polynucleotide molecule whose expression, or lack thereof, can be screened for or scored in some way. Marker genes for use in the practice of the present invention include, but are not limited to transcribable polynucleotide molecules encoding 13-glucuronidase (GUS, described in U.S. Pat. No. 5,599,670), green fluorescent protein and variants thereof (GFP, described in U.S. Pat. Nos. 5,491,084 and 6,146,826), proteins that confer antibiotic resistance, or proteins that confer herbicide tolerance. Useful antibiotic resistance markers, including those encoding proteins conferring resistance to kanamycin (nptll), hygromycin B (aph IV), streptomycin or spectinomycin (aad, spec/strep) and gentamycin (aac3 and aacC4), are well known in the art. Herbicides for which transgenic plant tolerance has been demonstrated and to which the method of the present invention can be applied, may include, but are not limited to: amino-methyl-phosphonic acid, glyphosate, glufosinate, sulfonylureas, imidazolinones, bromoxynil, dalapon, dicamba, cyclohexanedione, protoporphyrinogen oxidase inhibitors, and isoxasflutole herbicides. Transcribable polynucleotide molecules encoding proteins involved in herbicide tolerance are known in the art, and may include, but are not limited to, a transcribable polynucleotide molecule encoding 5-enolpyruvylshikimate-3-phosphate synthase (EPSPS for glyphosate tolerance, described in U.S. Pat. Nos. 5,627,061; 5,633,435; 6,040,497; and 5,094,945); a transcribable polynucleotide molecule encoding a glyphosate oxidoreductase and a glyphosate-N-acetyl transferase (GOX, described in U.S. Pat. No. 5,463,175; GAT, described in U.S. Patent Publication No. 20030083480; and dicamba monooxygenase, described in U.S. Patent Publication No. 20030135879); a transcribable polynucleotide molecule encoding bromoxynil nitrilase (Bxn for Bromoxynil tolerance, described in U.S. Pat. No. 4,810,648); a transcribable polynucleotide molecule encoding phytoene desaturase (crtI) described in Misawa, et al. (Plant Journal 4:833-840, 1993; and Plant Journal 6:481-489, 1994) for norflurazon tolerance; a transcribable polynucleotide molecule encoding acetohydroxyacid synthase (AHAS, aka ALS) described in Sathasiivan, et al. (Nucl. Acids Res. 18:2188-2193, 1990) for tolerance to sulfonylurea herbicides; and the bar gene described in DeBlock, et al. (EMBO Journal 6:2513-2519, 1987) for glufosinate and bialaphos tolerance. The promoter molecules of the present invention may express linked transcribable polynucleotide molecules that encode for phosphinothricin acetyltransferase, glyphosate resistant EPSPS, aminoglycoside phosphotransferase, hydroxyphenyl pyruvate dehydrogenase, hygromycin phosphotransferase, neomycin phosphotransferase, dalapon dehalogenase, bromoxynil resistant nitrilase, anthranilate synthase, aryloxyalkanoate dioxygenases, acetyl CoA carboxylase, glyphosate oxidoreductase, and glyphosate-N-acetyl transferase.

Included within the term “selectable markers” are also genes that encode a secretable marker whose secretion can be detected as a means of identifying or selecting for transformed cells. Examples include markers that encode a secretable antigen that can be identified by antibody interaction, or even secretable enzymes that can be detected catalytically. Selectable secreted marker proteins fall into a number of classes, including small, diffusible proteins which are detectable, (e.g. by ELISA), small active enzymes that are detectable in extracellular solution (e.g., α-amylase, β-lactamase, phosphinothricin transferase), or proteins that are inserted or trapped in the cell wall (such as proteins that include a leader sequence such as that found in the expression unit of extension or tobacco pathogenesis related proteins, also known as tobacco PRS). Other possible selectable marker genes will be apparent to those of skill in the art and are encompassed by the present invention.

Cell Transformation

The term “transformation” refers to the introduction of nucleic acid into a recipient host. As used herein, the term “host” refers to a bacterium, a fungus, or a plant, including any cells, tissue, organs, or progeny of the bacterium, fungus, or plant. For instance, a host cell according to the present invention may be any cell or organism, such as a plant cell, algae cell, algae, fungal cell, fungi, bacterial cell, insect cell, or the like. In an embodiment, hosts and transformed cells may include cells from: plants, Aspergillus, yeasts, insects, bacteria and algae. Plant tissues and cells of particular interest include, but are not limited to, protoplasts, calli, roots, tubers, seeds, stems, leaves, seedlings, embryos, and pollen.

As used herein, the term “transformed” refers to a cell, tissue, organ, or organism into which a foreign polynucleotide molecule, such as a construct, has been introduced. The introduced polynucleotide molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic organism as a parent in a cross and exhibiting an altered phenotype resulting from the presence of a foreign polynucleotide molecule. The term “transgenic” refers to a bacterium, fungus, or plant containing one or more heterologous polynucleic acid molecules.

There are many methods for introducing polynucleic acid molecules into plant cells. The method may generally comprise the steps of selecting a suitable host cell, transforming the host cell with a recombinant vector, and obtaining a transformed host cell. Suitable methods include bacterial infection (e.g. Agrobacterium), binary bacterial artificial chromosome vectors, direct delivery of DNA (e.g. via PEG-mediated transformation, desiccation/inhibition-mediated DNA uptake, electroporation, agitation with silicon carbide fibers, and acceleration of DNA coated particles, etc. (reviewed in Potrykus, et al., Ann. Rev. Plant Physiol. Plant Mol. Biol. 42: 205, 1991).

Technology for introduction of a DNA molecule into cells is well known to those of skill in the art. Methods and materials for transforming plant cells by introducing a plant DNA construct into a plant genome in the practice of this invention can include any of the well-known and demonstrated methods. Any transformation methods may be utilized to transform a host cell with one or more promoters and/or constructs of the present.

Regenerated transgenic plants can be self-pollinated to provide homozygous transgenic plants. Alternatively, pollen obtained from the regenerated transgenic plants may be crossed with non-transgenic plants, preferably inbred lines of agronomically important species. Descriptions of breeding methods that are commonly used for different traits and crops can be found in one of several reference books, see, for example, Allard, Principles of Plant Breeding, John Wiley & Sons, NY, U. of CA, Davis, Calif., 50-98, 1960; Simmonds, Principles of crop improvement, Longman, Inc., NY, 369-399, 1979; Sneep and Hendriksen, Plant breeding perspectives, Wageningen (ed), Center for Agricultural Publishing and Documentation, 1979; Fehr, Soybeans: Improvement, Production and Uses, 2nd Edition, Monograph, 16:249, 1987; Fehr, Principles of variety development, Theory and Technique, (Vol. 1) and Crop Species Soybean (Vol. 2), Iowa State Univ., Macmillan Pub. Co., NY, 360-376, 1987. Conversely, pollen from non-transgenic plants may be used to pollinate the regenerated transgenic plants.

The transformed plants may be analyzed for the presence of the genes of interest and the expression level and/or profile conferred by the regulatory elements of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays. The expression of a transcribable polynucleotide molecule can be measured using TaqMan® (Applied Biosystems, Foster City, Calif.) reagents and methods as described by the manufacturer and PCR cycle times determined using the TaqMan® Testing Matrix. Alternatively, the Invader® (Third Wave Technologies, Madison, Wis.) reagents and methods as described by the manufacturer can be used to evaluate transgene expression.

The seeds of plants of this invention may be harvested from fertile transgenic plants and used to grow progeny generations of transformed plants of this invention, including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immunoprecipitation, and ELISA.

Having now generally described the invention, the same will be more readily understood through reference to the following examples which are provided by way of illustration, and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

Sequence Listing

The attached sequence listing includes nucleic acid and amino acid sequences used in the work leading to the claimed invention.

SEQ ID NOS: 1 to 10 are nucleic acid sequences of mRNA sequences encoding squalene epoxidases.

SEQ ID NOS: 11 to 20 are the corresponding amino acid sequences.

SEQ ID NO: 21 is the amino acid sequence of RibB(G108S).

SEQ ID NO: 22 is the nucleic acid sequence of RibB(G108S) after Codon Optimization for Nicotiana tabacum (tobacco).

SEQ ID NO: 23 is the transit signal peptide (TP) sequence.

SEQ ID NO: 24 is a nucleic acid sequence of the SBPase cassette. The cassette contains DXPS, GO, MS, CAT, and SBPase, all fused with signal peptides for chloroplast expression and driven by strong constitutive promoters.

SEQ ID NOs: 25 and 26 are nucleic acid sequences for artificial microRNA targeting squalene epoxidase SQE3 sequences.

SEQ ID NOs: 27 and 28 are nucleic acid sequences for artificial microRNA targeting consensus sites of squalene epoxidase sequences of SQE1, SQE2 and SQE3.

In order to address various issues and advance the art, the entirety of this disclosure shows by way of illustration various embodiments in which the claimed invention(s) may be practiced and provide for superior production and yield of biological products. The advantages and features of the disclosure are of a representative sample of embodiments only, and are not exhaustive and/or exclusive. They are presented only to assist in understanding and teach the claimed features. It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope and/or spirit of the disclosure. Various embodiments may suitably comprise, consist of, or consist essentially of, various combinations of the disclosed elements, components, features, parts, steps, means, etc. In addition, the disclosure includes other inventions not presently claimed, but which may be claimed in future.

1. A genetic construct comprising a promoter and a coding sequence encoding one or more peptides, wherein expression of the one or more peptides leads to an increased yield of a biological product by:
reducing consumption of the bioproduct by reducing the activity of an enzyme that consumes the bioproduct; and/or
channeling carbon directly from photosynthesis to the production of 1-deoxy-D-xylulose 5-phosphate (DXP); and/or
increasing carbon fixation by photosynthesis.

2. The genetic construct of claim 1, wherein the bioproduct is selected from the group consisting of:
(i) a carbon-based bioproduct;
(ii) one or more terpenes; and
(iii) squalene.

3. (canceled)

4. (canceled)

5. The genetic construct of claim 1, wherein the consumption of the bioproduct is reduced by reducing the activity of squalene epoxidase.

6. The genetic construct of claim 5, wherein the construct encodes artificial microRNA which mediates squalene epoxidase knockdown, optionally wherein the artificial microRNA is amiRNA¹⁵⁹-SQE.

7. (canceled)

8. The genetic construct of claim 1, wherein the coding sequence encodes one or more further peptides, wherein expression of the one or more further peptides leads to an increased yield of the biological product by increasing the activity of squalene synthase (SQS) and/or farnesyl pyrophosphate synthase (FPS).

9. The genetic construct of claim 8, wherein the construct includes copies of the SQS or FPS encoding genes, or wherein the peptides cause overexpression of the SQS or FPS encoding genes.

10. (canceled)

11. The genetic construct of claim 1, wherein the coding sequence encodes one or more further peptides, wherein expression of the one or more further peptides leads to an increased yield of the biological product by signalling the transport of the bioproduct, optionally wherein the further peptide comprises a chloroplast transit peptide.

12. (canceled)

13. The genetic construct of claim 1, wherein carbon is channeled directly from photosynthesis to the production of 1-deoxy-D-xylulose 5-phosphate (DXP) by peptides that convert ribose-5-phosphate (R5P) or xylulose 5-phosphate (X5P) to DXP.

14. The genetic construct of claim 13, encoding a mutant RibB enzyme which converts R5P or X5P to DXP, or encoding RibB(G108S).

15. (canceled)

16. The genetic construct of claim 1, wherein carbon fixation by photosynthesis is increased by peptides that increase activity of the enzyme sedoheptulose-1,7-bisphosphatase (SBPase), optionally wherein the construct encodes SBPase.

17. (canceled)

18. A recombinant vector comprising the genetic construct of claim 1.

19. A method of increasing the yield of a biological product in a plant compared to the yield of the biological product in a wild-type plant cultured under the same conditions, the method comprising transforming a plant cell with the genetic construct of claim 1, and regenerating a plant from the transformed cell.

20. A method of producing a transgenic plant which produces a yield of a biological product which is higher than that of a corresponding wild-type plant cultured under the same conditions, the method comprising transforming a plant cell with the genetic construct of claim 1, and regenerating a plant from the transformed cell.

21. The method of claim 20, wherein the plant is a monocotyledonous plant, optionally selected from the group consisting of Oryza, Arundo, Hordeum, and Triticum, or wherein the plant is a dicotyledonous plant, optionally selected from the group consisting of Arabidopsis, Nicotiana, Lycopersicon, Glycine, Brassica, Vitis, Solanum, Manihot, Arachis, Malus, Citrus, Gossypium, Lactuca, and Raphanus.

22-24. (canceled)

25. A transgenic plant comprising the genetic construct of claim 1.

26. A host cell comprising the genetic construct of claim 1.

27. A plant propagation product obtainable from the transgenic plant of claim 25.

28. A biological product obtained from a modified plant comprising the genetic construct of claim 1, optionally wherein the biological product is a terpene or squalene.

29. (canceled)

30. (canceled)

31. A plant part containing higher levels of a biological product than a corresponding part of a wild-type plant cultured under the same conditions, wherein the plant part is harvested from the transgenic plant of claim 25.

32. A plant part of claim 31, wherein the plant part is a leaf.


SEQUENCE LISTING (28 sequences)

SEQ ID NO: 1 - DNA, 1842 bp, Nicotiana tabacum
atgctacttataaatatccccaacaatccacctttctccctcgtcttctccaaacacaattttcgtgtgtctacaaaccaaacacccccacaattatctcagccctctgcttcaagtaaaaagaaaaaattagccttgatctctttttccaaatatttgcgtaatacacagctccaattcatctctaccaacgagaaaatggtgaatttcatgatggataaatatattgtccccactttctttgtttctctattggggttccttcttctttatattttgcgaccaagattaagaaccggcccctattacaagaaaaaagaccctaaaaccagccaaaaatgtgacacccaaaatgtaatttctagcaatttaaccaatggtgaatgcaaattggaaaaagggtccgatgctgatatcatcattgttggagctggggttgctggtgctgctcttgctcatacccttgccaaggaagggcgaagagttcttgtcattgaaagggatttgacagagcccgatcggattgttggcgagttgctacagcctggaggttatcttaaattgattgagttgggccttgaagattgtgttgaggatattgatgcccagcgggtggttggatatgctcttttcaaggatgggaaaagcacaaatgtttcctatcccttggaaaatttccattctgatgtggctgggagaagcttccacaatgggcgtttcatacaaaagatgagagaaaaagcagctacttttcccaatgtacgattggagcaaggcactgtaacatccctgattgaagaaaatggatccgttaagggtatccagtacaaaacgaaggctggtcaagaacttaaagcacatgctcctcttacagtagtttgtgatgggtgcttttcaaacttgcgacgctctctttgcaaccctaaggttgatatcccatcttgctttgttggtttggtattggaactggagaacaatcaacttccatacccaaaccatgggcatgttattctggcagatccttcgcccatcttattttatcctattagtagcacagaaatccgctgcttggtcgatgtacctggtcaaaagcttccttctcttgctaatggtgatatggcaaattatttgaagactatggtggctccccaggtcccacctgagctacatgatgcttttataactgcaattgataaggggcatattagaactatgccaaataggagcatgccagctgctccgtatcctacccctggagctctgttacttggtgattctttcaacatgcgtcatcctttaactggtgggggaatgacagttgcgctttcagatattgcagtgttaaggaatcttcttaagccattgaacgacctgaatgatgcagatgagctatgtaaatatctggagtccttttatactttgcgcaagccagtggcttcaacaataaatactttggctggagcactgtacaaggtgttctgtgcttctcctgatcaagcgaggaaggagatgcgagaagcatgtttcgactatttgagtcttggaggtacttgttcaacaggacccgtagctctactctctggtcttaatcctagcccgctgagcttggtactccatttctttgctgtggccatatatggagttggtcgtttactcgttccatttccttccccaaagagattgtggattggagctagattaatctcggctgcatcgagtatcatatttcccattataaaagcagaaggggtcaggcaaatgttcttcccaacaacaataccagcatatcacagagctcctccagtaaacaaggggtcagatcagattaagcaaaataaataa

SEQ ID NO: 2 - DNA, 1821 bp, Nicotiana tabacum
atgctgcttataaatactcccaacaatatatctttctccctcgtcttctccaaacacaaatttcctgtgtcaacaaaccaaacacccccacaattatctcagccctctgcttcaagtacaaagaaaaaattagccttgatctctttttccaaatatttgcgaaatacacagctccaatttatctctaccaacgagaaaatggtgaatttcatgatggataaatatattgtccccactttctttgtttctttattggggttccttcttctttatattttgcgacctagatttagaactccctattacaagaaaaaagaccctaaatccatccaaaaatgtgaaacccacaatgtgatttctagcaatttaatcaatggtgaatgcaaattggaaaaagggtccgacgctgatatcatcattgttggagctggggttgctggtgctgctcttgctcatacccttgccaaggaagggcgaaaagttcttgtacttgaaagggatttgacagagcccgaccggattgttggcgagttgctacagcctggaggttatctgaaattgattgagttgggccttgaagattgtgttgaggatattgatgcccagcgggtggttggatatgctcttttcaaggatgggaaaagcacaaacgtttcctatcccttggaaaatttccattctgatgtggctgggagaagctttcacaatgggcgtttcatacaaaagatgagagaaaaagcagctactcttcccaatgtacgattggagcaaggcactgtaacatccctgattgaagaaaatggatccgttaagggtgtccagtacaaaacgaaggctggtcaagaacttaaagcacatgctcctcttacagtagtttgtgatggatgcttttcaaacttgcgacgctcgctttgcaaccctaaggttgatattccctcttgttttgttggtttggtattggagctagagaatgatcaacttccatacccaaaccatgggcatgttattctggcagatccttcgcccatcttattttatcctattagtagcacggaaatccgctgcttggtcgatgtgcctggtcaaaagcttccttctcttgctaatggtgatatggcaaattatttgaagactatggtggctccccaggtcccacctgagctacatgatgcgtttataactgcaattgataaggggcatattagaactatgccaaataggagcatgccagctgctccgtatcctacccctggagctctgttacttggtgattctttcaacatgcgccatcctttaactggtgggggaatgactgtagcgctttcagatattgcagtgttaaggaatcttcttaagccattggacaacctgaatgatgcagatgagttatgtaaatatttggagtccttttatactttgcgcaagccagtggcttcaacaataaatactttggctggagcactgtacaaggtattctgtgcttctcctgatcaagcgaggaaggagatgcgagaagcatgtttcgactatttgagtcttggaggtacttgttcaacaggacccgtagctctactctctggtcttaatcctagcccgctgagcttggtactccatttttttgctgtggccatatatggagttggtcgtttactcgttccatttccttccccaaagagattgtggataggagctagattaatctcggctgcatcgggcatcatatttcccattataaaagcagaaggggtcaggcaaatgttcttcccaacaacaataccagcatatcacagagctcctccagtaaacaaggggtcagattaa

SEQ ID NO: 3 - DNA, 1587 bp, Nicotiana tabacum
atggtggatatgggtctttccggatctgtacttgcggtggtgtttggcttcgtggcagttttgtggtttttcattcagagaaatggtggccgcaaagataaaaataatgaccccgccaccaccagcaccactaccacaacggtgtatgatggagaatgcagatcaaaggacgcaaacgatgacgctgacatcatcatcgtcggtgctggcgttgccggtgccgctcttgctcacacccttggcaaagaggggcgtcatgtaaaagtgattgaaagagatttgacagagcctgatcgaattgttggagaactcctacaaccaggtggcttcctcaaattgcaggagttgggtttggaagattgtgtggagaatattgatgctcagcgagtgttcgggtatgctcttttcaaggatggcaagagcactcgtctttcttatcccctggagaagtttcatgctgatgtatctgggaggagctttcacaatgggcgtttcattcaaagaatgagagagaaagctgcatctcttcccaatgtgaaactggagcaaggcactgttacgtctctgctagaagaaaacgggaccattagaggtgttcagtacaaaaacaaatctggtgaagagttgaaagcctatgcaccattgaccatagtgtgcgatggttgtttctcgaatctacggcgtacactttgtgacccaaaggtagaagtgccttcttgttttgttggtctggtcctggagaactgccagcttccacatgcaaaccatggacatgtcattttggctgatccatcacctatcctgttctaccccataagcagtactgaggtccgttgccttgttgatgtacctggtcaaaaagtgccttctatttcaaatggtgaaatggccaaatatttgaaaagcgtcgtcgctccccaggtgcctcctgagataaaagatgcattcattgctgcaattgataaagggaacatcagaacaatgccaaaccgaagcatgccagctgctcctcatccaactcctggtgctcttctcatgggagatgcattcaacatgcgccatcccttgactggtggaggaatgactgtagcattgtctgatattgttgtattacgtgatcttcttaaacctcttcgtgatttgaatgatgcacctactctttgtagatatctggagtccttttacaccttgcgtaagccggtggcatctaccatcaatacattggctggtgcattgtataaggtgttttgtgcttcacctgatcaagctaggaaggagatgcgcgatgcatgctttgactatttgagccttggtggagtattctccacaggacctgtatctttgctttctggcttaaaccctcgtccattaagtctcgtttgtcacttctttgctgtggctatctatggtgttggccgcttgctgctgcctttcccatcacccaaacgtatgtggattggagcccggctgatctcgggtgcatctggaattattttccctatcatcaaggcagaaggagtgaggcagatgttcttccccgctactgttcctgcatattacaggtctcctcctgaagtgaaattgtaa

SEQ ID NO: 4 - DNA, 1593 bp, Nicotiana tabacum
atggtggatatgggtctttccggatctgtacttgcggtggtgtttggcttcgtggcagttttgtggtttttcattcagagaaacggtggccgcaaagataaaaataatgaccccgccaccgccaccaccagcaccactacaacaacggtgtatgatggagaatgcagatcaaaggatgcaaacgacaacgctgacatcatcatcgtcggtgccggagttgccggtgccgctcttgctcacacccttggcaaagaggggcgtcgtgtaaaagtaattgaaagagatttgacagagcctgatcgaattgttggagaactcctacaaccaggtggcttcctcaaattgcaggagttgggtttggaagattgtgtggagaatattgatgctcagcgagtgtttgggtatgctcttttcaaggatggcaagagcactcgtctttcttatcccctggagaaatttcacgctgaggtttctggaaggagctttcacaatgggcgtttcattcaaagaatgcgagagaaagctgcatctcttcccaatgtcaaactggagcaaggcactgttacgtctctgcttgaagaaaacgggaccattagaggtgttcagtacaaaaacaaatctggcgaagagttgaaagcctatgcaccattgaccatagtatgtgatggttgtttctcaaatctacggcgtaccctttgcgacccaaaggtagaagtgccttcctgttttgttggtctggtcctggagaactgccagcttccacatgcaaaccatggacatgtcattttggctgatccatcacctatcttgttctatcccataagcagtaccgaggtccgctgtctggttgatgtacctggtcaaaaagtgccttctatttcaaatggtgaaatgggcaaatatttgaaaagcatcgttgctccccaggtccctcctgagataaaagatgcattcattgccgcaattgataaaggtaacatcaggacaatgccaaaccgaagcatgccagctgctcctcatccaactcctggtgctcttctcatgggagatgcattcaatatgcgtcatcccttgactggtggaggaatgactgtagcattgtctgatattgttgtattacgtgatcttcttaaacctcttcgtgatttgaatgatgcacctacactttgtagatatctggagtccttttacaccttgcgtaagcctgtggcatccaccataaatacattggctggtgccttgtataaggtgttttgtgcttcacctgatcaagctaggaaggaaatgcgcgatgcatgctttgactatttgagcctcggtggagtattctcaacaggaccagtatctttgctttctggcttaaaccctcgtccattaagccttgtttgtcatttctttgctgtggcaatctatggtgttggccgcttgctgctgcctttcccatcacccaaacgtatgtggattggagcccggctgatatcgggtgcatctggaatcattttcccgatcatcaaagcagaaggagtgaggcagatgttcttccctgctactgttcctgcatattacaggtctcctcctgaagtgaaattgtaa

SEQ ID NO: 5 - DNA, 1266 bp, Nicotiana tabacum
atggtggatatgggtctttccggatctgtacttgcggtggtgtttggcttcgtggcagttttgtggtttttcattcagagaaatggtggccgcaaagataaaaataatgaccccgccaccaccagcaccactaccacaacggtgtatgatggagaatgcagatcaaaggacgcaaacgatgacgctgacatcatcatcgtcggtgctggcgttgccggtgccgctcttgctcacacccttggcaaagaggggcgtcatgtaaaagtgattgaaagagatttgacagagcctgatcgaattgttggagaactcctacaaccaggtggcttcctcaaattgcaggagttgggtttggaagattgtgtggagaatattgatgctcagcgagtgttcgggtatgctcttttcaaggatggcaagagcactcgtctttcttatcccctggagaagtttcatgctgatgtatctgggaggagctttcacaatgggcgtttcattcaaagaatgagagagaaagctgcatctcttcccaatgtgaaactggagcaaggcactgttacgtctctgctagaagaaaacgggaccattagaggtgttcagtacaaaaacaaatctggtgaagagttgaaagcctatgcaccattgaccatagtgtgcgatggttgtttctcgaatctacggcgtacactttgtgacccaaaggtagaagtgccttcttgttttgttggtctggtcctggagaactgccagcttccacatgcaaaccatggacatgtcattttggctgatccatcacctatcctgttctaccccataagcagtactgaggtccgttgccttgttgatgtacctggtcaaaaagtgccttctatttcaaatggtgaaatggccaaatatttgaaaagcgtcgtcgctccccaggtgcctcctgagataaaagatgcattcattgctgcaattgataaagggaacatcagaacaatgccaaaccgaagcatgccagctgctcctcatccaactcctggtgctcttctcatgggagatgcattcaacatgcgccatcccttgactggtggaggaatgactgtagcattgtctgatattgttgtattacgtgatcttcttaaacctcttcgtgatttgaatgatgcacctactctttgtagatatctggagtccttttacaccttgcgtaaggtaaatatcattttaaatgtttcctcccataagcagaagatttggagctggtacctattatctacttaa

SEQ ID NO: 6 - DNA, 1305 bp, Nicotiana tabacum
atggtggatatgggtctttccggatctgtacttgcggtggtgtttggcttcgtggcagttttgtggtttttcattcagagaaacggtggccgcaaagataaaaataatgaccccgccaccgccaccaccagcaccactacaacaacggtgtatgatggagaatgcagatcaaaggatgcaaacgacaacgctgacatcatcatcgtcggtgccggagttgccggtgccgctcttgctcacacccttggcaaagaggggcgtcgtgtaaaagtaattgaaagagatttgacagagcctgatcgaattgttggagaactcctacaaccaggtggcttcctcaaattgcaggagttgggtttggaagattgtgtggagaatattgatgctcagcgagtgtttgggtatgctcttttcaaggatggcaagagcactcgtctttcttatcccctggagaaatttcacgctgaggtttctggaaggagctttcacaatgggcgtttcattcaaagaatgcgagagaaagctgcatctcttcccaatgtcaaactggagcaaggcactgttacgtctctgcttgaagaaaacgggaccattagaggtgttcagtacaaaaacaaatctggcgaagagttgaaagcctatgcaccattgaccatagtatgtgatggttgtttctcaaatctacggcgtaccctttgcgacccaaaggtagaagtgccttcctgttttgttggtctggtcctggagaactgccagcttccacatgcaaaccatggacatgtcattttggctgatccatcacctatcttgttctatcccataagcagtaccgaggtccgctgtctggttgatgtacctggtcaaaaagtgccttctatttcaaatggtgaaatgggcaaatatttgaaaagcatcgttgctccccaggtccctcctgagataaaagatgcattcattgccgcaattgataaaggtaacatcaggacaatgccaaaccgaagcatgccagctgctcctcatccaactcctggtgctcttctcatgggagatgcattcaatatgcgtcatcccttgactggtggaggaatgactgtagcattgtctgatattgttgtattacgtgatcttcttaaacctcttcgtgatttgaatgatgcacctacactttgtagatatctggagtccttttacaccttgcgtaaggtaaaaatcattttaaatctttcctcccataaacaaaagatttggagccggtacctgttttctactttaagaatccctagtgaagtaagagctctatgttga

SEQ ID NO: 7 - DNA, 1551 bp, Nicotiana tabacum
atggagttggtaattctgttcatcgcttccctttttggactcctcttgctgtacaatatgatcagaaggaagataatggaaataagaaacataacatcaaatgtggtagtgatgaaggatcataaatcctatggtaatacggatgttatcattgtcggtgccggcgttgctggttctgctttggcctgcactcttggcaaggacgggcgacgagttcacgtgatagagagggacttgactgagccagacagaatggttggtgaacttttacagccaggtggctatctcaaattagttgagttaggccttgaagattgcttggaagatatagatgctcaaagagttggaggacatgttctttacaagaagaatggagaacatattatgctttcctatcccttagagaagttccaggcagtagatgcatcggggagatgtttccacaatggacgtttcgttcagaagatgagggaaaaagctgccaccctccccaatgtaagacttgaaaagggaacagtcacgtctttgattgaagaaaagggaagtgtgaaaggagtaaattacaagacaaaagatggacgagagctaactgcttatgctcctcttacaattgtttgcgatggttgcttctcaaatttgcgacgctccttatgcaatcccaagatggatatcccttctacttatgttggattaattctaaaagattgtcaactcccatatgcaaaccacggagttcttgtcatgtcagacccatcaccagtaacattttatccaatcagcagcactgagattcgttgtctggttcttattgctggtcaaaaagtaccttccatcgctaatggagaaatggtcaattatttgaagaatgtggtcgcttctcaggtacctagtgaattgagggatgcattaataagagcgattggcgaggaaaacaacataagaagaatgcaattcagaagcatggcagcaaatacttgtacaattccaggttctcttctaataggagacgccttcaatatgaggcatcctttatcagctggaggaatgactgtcgctctttccgacattgttgtccttcgccatcttcttaaacctctccctaattttactgatgcaaacgctgttcttcaacgcttttacactcttagaaaaccggttgcatcaacaataaacatattggctggtgtaacataccaagttttcagtccttcacttgatccatcaagaaaagagattagggaagcattttatggttatttaaggcttggagggatttttacaaatggagcaattggtttacttgctggcctaaatccaacgcctctcggcttcgcttttcaattcattgctgtggcagtatatacttttggtcgattattgcttccgtttcctactcccaacgccattttgcgcggggccagattgctttatgtcgcaattgatatgatattccccattattaaagcagaaggaattagacaaatgttcttcccagctagccttccagcctactatagtgcacctgcattagctcataaaagtgaccaataa

SEQ ID NO: 8 - DNA, 1530 bp, Nicotiana tabacum
atggagatgataatttttttgggcagttttatcgcttccctttttggactcctcttgctgtacaatatgatcagaaggaaaataatggaaataagaaacatagcatcaaatgtggtactgattaaggatcataagtcctatggtaaaacggatgttatcattgtcggtgccggcgttgctggttctgctttggcctgcacttttggtaaggatggacgacgagttcacgtgatagagagggacttgactgaaccacatagaatggttggtgaacttctacagccaggtggctatctcaaattagtcgacttaggccttgaagattgcttggaagatatagatgctcaaagagttgtaggatatactctttacaagaagaatggagaacatattatgctttcctatcccttagagaagttccaggcagcagatatgtccggaagatgtttccacaatgggcgtttcgttcagaagatgagggaaaaagctgccaccctccccaatgtaagacttgaacagggaacagtcacgtctttgatagaagaaaagggaactgtgaaaggggtaaattacaagacaaaagatggacgagagctatctgcttatgctcctcttacaattgtttgtgatggttgcttctcgaatctgcgacgctccttatgcaatctcaagatggatattccttcgacttatgttggattaattttaaaagattgtcaactcccatatgcaaaccacggagttcttgtcatgtcagacccttcaccagtgacattttatccagtcagcagcactgaggttcgttgtctggttcttattgctggtcaaaatgtaccttctatcgctaatggagaaatggtcaattatttgacgaatgtggttgctcctcaggtacctagtgaattgagggatgcattaataacagcgattagcgaggaaaacaatataagaacaatgcaattcagaagcatgccagcaacttgtagtacaattccaggttctcttctaataggagatgccttcaatatgaggcatcctttatcagcaggaggaatgactgttgctctttccgacattgttgtccttcgccatcttcttaaacctctcactgactttaatgatgcaaatgctgtgtccaagtatcttcaacgcttttacactcttagaaaaccactagcatcaacaataaacatactggctggtgtaacataccaagttttcagtccttcgcttgatccatcaagaaaagagattagggaagtattttatggttatttaagagttggagggatttttacaaatggagcaattggtttacttgctggtctaaatccaaggcctctcagcttgccttttcatttcattgctgtggcagtatatacttttggtcgcttattgcttccatttcctactcccaagggaattttgcgcgctgccacgttgctttatgttgcatgtggtatgatatttcccattattaaagcagaaggaattaaacacatgttttttccagcaatttttccagcctag

SEQ ID NO: 9 - DNA, 1260 bp, Nicotiana tabacum
atggagatgataatttttttgggcagttttatcgcttccctttttggactcctcttgctgtacaatatgatcagaaggaaaataatggaaataagaaacatagcatcaaatgtggtactgattaaggatcataagtcctatggtaaaacggatgttatcattgtcggtgccggcgttgctggttctgctttggcctgcacttttggtaaggatggacgacgagttcacgtgatagagagggacttgactgaaccacatagaatggttggtgaacttctacagccaggtggctatctcaaattagtcgacttaggccttgaagattgcttggaagatatagatgctcaaagagttgtaggatatactctttacaagaagaatggagaacatattatgctttcctatcccttagagaagttccaggcagcagatatgtccggaagatgtttccacaatgggcgtttcgttcagaagatgagggaaaaagctgccaccctccccaatgtaagacttgaacagggaacagtcacgtctttgatagaagaaaagggaactgtgaaaggggtaaattacaagacaaaagatggacgagagctatctgcttatgctcctcttacaattgtttgtgatggttgcttctcgaatctgcgacgctccttatgcaatctcaagatggatattccttcgacttatgttggattaattttaaaagattgtcaactcccatatgcaaaccacggagttcttgtcatgtcagacccttcaccagtgacattttatccagtcagcagcactgaggttcgttgtctggttcttattgctggtcaaaatgtaccttctatcgctaatggagaaatggtcaattatttgacgaatgtggttgctcctcaggtacctagtgaattgagggatgcattaataacagcgattagcgaggaaaacaatataagaacaatgcaattcagaagcatgccagcaacttgtagtacaattccaggttctcttctaataggagatgccttcaatatgaggcatcctttatcagcaggaggaatgactgttgctctttccgacattgttgtccttcgccatcttcttaaacctctcactgactttaatgatgcaaatgctgtgtccaagtatcttcaacgcttttacactcttagaaaatatatacttttggtcgcttattgcttccatttcctactcccaagggaattttgcgcgctgccacgttgctttatgttgcatgtggtatga

SEQ ID NO: 10 - DNA, 888 bp, Nicotiana tabacum
atggatatcccttctacttatgttggattaattctaaaagattgtcaactcccatatgcaaaccacggagttcttgtcatgtcagacccatcaccagtaacattttatccaatcagcagcactgagattcgttgtctggttcttattgctggtcaaaaagtaccttccatcgctaatggagaaatggtcaattatttgaagaatgtggtcgcttctcaggtacctagtgaattgagggatgcattaataagagcgattggcgaggaaaacaacataagaagaatgcaattcagaagcatggcagcaaatacttgtacaattccaggttctcttctaataggagacgccttcaatatgaggcatcctttatcagctggaggaatgactgtcgctctttccgacattgttgtccttcgccatcttcttaaacctctccctaattttactgatgcaaacgctgttcttcaacgcttttacactcttagaaaaccggttgcatcaacaataaacatattggctggtgtaacataccaagttttcagtccttcacttgatccatcaagaaaagagattagggaagcattttatggttatttaaggcttggagggatttttacaaatggagcaattggtttacttgctggcctaaatccaacgcctctcggcttcgcttttcaattcattgctgtggcagtatatacttttggtcgattattgcttccgtttcctactcccaacgccattttgcgcggggccagattgctttatgtcgcaattgatatgatattccccattattaaagcagaaggaattagacaaatgttcttcccagctagccttccagcctactatagtgcacctgcattagctcataaaagtgaccaataa

SEQ ID NO: 11 - protein, 516 aa, Nicotiana tabacum
MELVILFIASLFGLLLLYNMIRRKIMEIRNITSNVVVMKDHKSYGNTDVIIVGAGVAGSALACTLGKDGRRVHVIERDLTEPDRMVGELLQPGGYLKLVELGLEDCLEDIDAQRVGGHVLYKKNGEHIMLSYPLEKFQAVDASGRCFHNGRFVQKMREKAATLPNVRLEKGTVTSLIEEKGSVKGVNYKTKDGRELTAYAPLTIVCDGCFSNLRRSLCNPKMDIPSTYVGLILKDCQLPYANHGVLVMSDPSPVTFYPISSTEIRCLVLIAGQKVPSIANGEMVNYLKNVVASQVPSELRDALIRAIGEENNIRRMQFRSMAANTCTIPGSLLIGDAFNMRHPLSAGGMTVALSDIVVLRHLLKPLPNFTDANAVLQRFYTLRKPVASTINILAGVTYQVFSPSLDPSRKEIREAFYGYLRLGGIFTNGAIGLLAGLNPTPLGFAFQFIAVAVYTFGRLLLPFPTPNAILRGARLLYVAIDMIFPIIKAEGIRQMFFPASLPAYYSAPALAHKSDQ

SEQ ID NO: 12 - protein, 295 aa, Nicotiana tabacum
MDIPSTYVGLILKDCQLPYANHGVLVMSDPSPVTFYPISSTEIRCLVLIAGQKVPSIANGEMVNYLKNVVASQVPSELRDALIRAIGEENNIRRMQFRSMAANTCTIPGSLLIGDAFNMRHPLSAGGMTVALSDIVVLRHLLKPLPNFTDANAVLQRFYTLRKPVASTINILAGVTYQVFSPSLDPSRKEIREAFYGYLRLGGIFTNGAIGLLAGLNPTPLGFAFQFIAVAVYTFGRLLLPFPTPNAILRGARLLYVAIDMIFPIIKAEGIRQMFFPASLPAYYSAPALAHKSDQ

SEQ ID NO: 13 - protein, 509 aa, Nicotiana tabacum
MEMIIFLGSFIASLFGLLLLYNMIRRKIMEIRNIASNVVLIKDHKSYGKTDVIIVGAGVAGSALACTFGKDGRRVHVIERDLTEPHRMVGELLQPGGYLKLVDLGLEDCLEDIDAQRVVGYTLYKKNGEHIMLSYPLEKFQAADMSGRCFHNGRFVQKMREKAATLPNVRLEQGTVTSLIEEKGTVKGVNYKTKDGRELSAYAPLTIVCDGCFSNLRRSLCNLKMDIPSTYVGLILKDCQLPYANHGVLVMSDPSPVTFYPVSSTEVRCLVLIAGQNVPSIANGEMVNYLTNVVAPQVPSELRDALITAISEENNIRTMQFRSMPATCSTIPGSLLIGDAFNMRHPLSAGGMTVALSDIVVLRHLLKPLTDFNDANAVSKYLQRFYTLRKPLASTINILAGVTYQVFSPSLDPSRKEIREVFYGYLRVGGIFTNGAIGLLAGLNPRPLSLPFHFIAVAVYTFGRLLLPFPTPKGILRAATLLYVACGMIFPIIKAEGIKHMFFPAIFPA

SEQ ID NO: 14 - protein, 419 aa, Nicotiana tabacum
MEMIIFLGSFIASLFGLLLLYNMIRRKIMEIRNIASNVVLIKDHKSYGKTDVIIVGAGVAGSALACTFGKDGRRVHVIERDLTEPHRMVGELLQPGGYLKLVDLGLEDCLEDIDAQRVVGYTLYKKNGEHIMLSYPLEKFQAADMSGRCFHNGRFVQKMREKAATLPNVRLEQGTVTSLIEEKGTVKGVNYKTKDGRELSAYAPLTIVCDGCFSNLRRSLCNLKMDIPSTYVGLILKDCQLPYANHGVLVMSDPSPVTFYPVSSTEVRCLVLIAGQNVPSIANGEMVNYLTNVVAPQVPSELRDALITAISEENNIRTMQFRSMPATCSTIPGSLLIGDAFNMRHPLSAGGMTVALSDIVVLRHLLKPLTDFNDANAVSKYLQRFYTLRKYILLVAYCFHFLLPREFCALPRCFMLHVV

SEQ ID NO: 15 - protein, 528 aa, Nicotiana tabacum
MVDMGLSGSVLAVVFGFVAVLWFFIQRNGGRKDKNNDPATTSTTTTTVYDGECRSKDANDDADIIIVGAGVAGAALAHTLGKEGRHVKVIERDLTEPDRIVGELLQPGGFLKLQELGLEDCVENIDAQRVFGYALFKDGKSTRLSYPLEKFHADVSGRSFHNGRFIQRMREKAASLPNVKLEQGTVTSLLEENGTIRGVQYKNKSGEELKAYAPLTIVCDGCFSNLRRTLCDPKVEVPSCFVGLVLENCQLPHANHGHVILADPSPILFYPISSTEVRCLVDVPGQKVPSISNGEMAKYLKSVVAPQVPPEIKDAFIAAIDKGNIRTMPNRSMPAAPHPTPGALLMGDAFNMRHPLTGGGMTVALSDIVVLRDLLKPLRDLNDAPTLCRYLESFYTLRKPVASTINTLAGALYKVFCASPDQARKEMRDACFDYLSLGGVFSTGPVSLLSGLNPRPLSLVCHFFAVAIYGVGRLLLPFPSPKRMWIGARLISGASGIIFPIIKAEGVRQMFFPATVPAYYRSPPEVKL

SEQ ID NO: 16 - protein, 530 aa, Nicotiana tabacum
MVDMGLSGSVLAVVFGFVAVLWFFIQRNGGRKDKNNDPATATTSTTTTTVYDGECRSKDANDNADIIIVGAGVAGAALAHTLGKEGRRVKVIERDLTEPDRIVGELLQPGGFLKLQELGLEDCVENIDAQRVFGYALFKDGKSTRLSYPLEKFHAEVSGRSFHNGRFIQRMREKAASLPNVKLEQGTVTSLLEENGTIRGVQYKNKSGEELKAYAPLTIVCDGCFSNLRRTLCDPKVEVPSCFVGLVLENCQLPHANHGHVILADPSPILFYPISSTEVRCLVDVPGQKVPSISNGEMGKYLKSIVAPQVPPEIKDAFIAAIDKGNIRTMPNRSMPAAPHPTPGALLMGDAFNMRHPLTGGGMTVALSDIVVLRDLLKPLRDLNDAPTLCRYLESFYTLRKPVASTINTLAGALYKVFCASPDQARKEMRDACFDYLSLGGVFSTGPVSLLSGLNPRPLSLVCHFFAVAIYGVGRLLLPFPSPKRMWIGARLISGASGIIFPIIKAEGVRQMFFPATVPAYYRSPPEVKL

SEQ ID NO: 17 - protein, 421 aa, Nicotiana tabacum
MVDMGLSGSVLAVVFGFVAVLWFFIQRNGGRKDKNNDPATTSTTTTTVYDGECRSKDANDDADIIIVGAGVAGAALAHTLGKEGRHVKVIERDLTEPDRIVGELLQPGGFLKLQELGLEDCVENIDAQRVFGYALFKDGKSTRLSYPLEKFHADVSGRSFHNGRFIQRMREKAASLPNVKLEQGTVTSLLEENGTIRGVQYKNKSGEELKAYAPLTIVCDGCFSNLRRTLCDPKVEVPSCFVGLVLENCQLPHANHGHVILADPSPILFYPISSTEVRCLVDVPGQKVPSISNGEMAKYLKSVVAPQVPPEIKDAFIAAIDKGNIRTMPNRSMPAAPHPTPGALLMGDAFNMRHPLTGGGMTVALSDIVVLRDLLKPLRDLNDAPTLCRYLESFYTLRKVNIILNVSSHKQKIWSWYLLST

SEQ ID NO: 18 - protein, 434 aa, Nicotiana tabacum
MVDMGLSGSVLAVVFGFVAVLWFFIQRNGGRKDKNNDPATATTSTTTTTVYDGECRSKDANDNADIIIVGAGVAGAALAHTLGKEGRRVKVIERDLTEPDRIVGELLQPGGFLKLQELGLEDCVENIDAQRVFGYALFKDGKSTRLSYPLEKFHAEVSGRSFHNGRFIQRMREKAASLPNVKLEQGTVTSLLEENGTIRGVQYKNKSGEELKAYAPLTIVCDGCFSNLRRTLCDPKVEVPSCFVGLVLENCQLPHANHGHVILADPSPILFYPISSTEVRCLVDVPGQKVPSISNGEMGKYLKSIVAPQVPPEIKDAFIAAIDKGNIRTMPNRSMPAAPHPTPGALLMGDAFNMRHPLTGGGMTVALSDIVVLRDLLKPLRDLNDAPTLCRYLESFYTLRKVKIILNLSSHKQKIWSRYLFSTLRIPSEVRALC

SEQ ID NO: 19 - protein, 613 aa, Nicotiana tabacum
MLLINIPNNPPFSLVFSKHNFRVSTNQTPPQLSQPSASSKKKKLALISFSKYLRNTQLQFISTNEKMVNFMMDKYIVPTFFVSLLGFLLLYILRPRLRTGPYYKKKDPKTSQKCDTQNVISSNLTNGECKLEKGSDADIIIVGAGVAGAALAHTLAKEGRRVLVIERDLTEPDRIVGELLQPGGYLKLIELGLEDCVEDIDAQRVVGYALFKDGKSTNVSYPLENFHSDVAGRSFHNGRFIQKMREKAATFPNVRLEQGTVTSLIEENGSVKGIQYKTKAGQELKAHAPLTVVCDGCFSNLRRSLCNPKVDIPSCFVGLVLELENNQLPYPNHGHVILADPSPILFYPISSTEIRCLVDVPGQKLPSLANGDMANYLKTMVAPQVPPELHDAFITAIDKGHIRTMPNRSMPAAPYPTPGALLLGDSFNMRHPLTGGGMTVALSDIAVLRNLLKPLNDLNDADELCKYLESFYTLRKPVASTINTLAGALYKVFCASPDQARKEMREACFDYLSLGGTCSTGPVALLSGLNPSPLSLVLHFFAVAIYGVGRLLVPFPSPKRLWIGARLISAASSIIFPIIKAEGVRQMFFPTTIPAYHRAPPVNKGSDQIKQNK

SEQ ID NO: 20 - protein, 606 aa, Nicotiana tabacum
MLLINTPNNISFSLVFSKHKFPVSTNQTPPQLSQPSASSTKKKLALISFSKYLRNTQLQFISTNEKMVNFMMDKYIVPTFFVSLLGFLLLYILRPRFRTPYYKKKDPKSIQKCETHNVISSNLINGECKLEKGSDADIIIVGAGVAGAALAHTLAKEGRKVLVLERDLTEPDRIVGELLQPGGYLKLIELGLEDCVEDIDAQRVVGYALFKDGKSTNVSYPLENFHSDVAGRSFHNGRFIQKMREKAATLPNVRLEQGTVTSLIEENGSVKGVQYKTKAGQELKAHAPLTVVCDGCFSNLRRSLCNPKVDIPSCFVGLVLELENDQLPYPNHGHVILADPSPILFYPISSTEIRCLVDVPGQKLPSLANGDMANYLKTMVAPQVPPELHDAFITAIDKGHIRTMPNRSMPAAPYPTPGALLLGDSFNMRHPLTGGGMTVALSDIAVLRNLLKPLDNLNDADELCKYLESFYTLRKPVASTINTLAGALYKVFCASPDQARKEMREACFDYLSLGGTCSTGPVALLSGLNPSPLSLVLHFFAVAIYGVGRLLVPFPSPKRLWIGARLISAASGIIFPIIKAEGVRQMFFPTTIPAYHRAPPVNKGSD

SEQ ID NO: 21 - protein, 217 aa, Artificial Sequence
  RibB(G108S) protein sequence
MNQTLLSSFGTPFERVENALAALREGRGVMVLDDEDRENEGDMIFPAETMTVEQMALTIRHGSGIVCLCITEDRRKQLDLPMMVENNTSAYGTGFTVTIEAAEGVTTSVSAADRITTVRAAIADGAKPSDLNRPGHVFPLRAQAGGVLTRGGHTEATIDLMTLAGFKPAGVLCELTNDDGTMARAPECIEFANKHNMALVTIEDLVAYRQAHERKAS

SEQ ID NO: 22 - DNA, 654 bp, Artificial Sequence
  RibB(G108S) DNA sequence after Codon Optimization for Nicotiana tabacum
atgaatcaaactctattatcgtcttttgggacgccgttcgagagagtcgagaacgccttagctgcattaagggaaggaagaggtgtcatggtgcttgacgacgaagatagagaaaacgaaggagacatgatctttccggcggaaacgatgactgtggaacaaatggcattgacaattagacatgggtccggtattgtatgtttgtgtatcacagaagatagaaggaaacagttagatctgcctatgatggtggagaataatacttctgcatacggtaccggttttactgtaactatagaggcagctgagggtgtcacaacatcagtctctgctgcggataggataactacggtccgagctgcaattgctgatggtgctaaaccttcagacttgaatcgacccgggcatgtatttcctttaagggcgcaagccggtggtgttttgactcgagggggacacactgaggcaacaattgacttgatgactttagcaggattcaaaccggcgggagtcctttgtgaattgacaaatgacgacggaacgatggccagggcacctgagtgcatcgagttcgccaacaaacataatatggctttagtgaccatagaagacttagttgcatacaggcaggcccatgaacgaaaagcaagttga

SEQ ID NO: 23 - DNA, 210 bp, Artificial Sequence
  (Chloroplast) Transit peptide sequence
atggcttcctctatgctctcctccgccgctgtggttacatccccggctcaggccaccatggtcgctccattcaccggcttgaagtcatccgctgcattcccggtcacccgcaagaccaacaaggacatcacttccatcgcaagcaacgggggaagatctagctgcatgaagactagtatgcagccccatcatcatcataaagaggggcgt

SEQ ID NO: 24 - DNA, 2287 bp, Artificial Sequence
  Nucleic acid sequence of the SBPase cassette
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgtctagaggatccatggagaccagcatcgcgtgctactcacgtgggatccttcccccaagtgtctcttctcaacgatcctctacattggtctctcctccttcctactccacatcctccagcttcaagcgtctaaaatcgagctcaatcttcggagattcactacgattagcaccaaaatcgcaacttaaagccacaaaagctaagagcaatggtgcttcaactgtgaccaaatgtgaaattggccaaagcttggaagagtttttggcacaagcaactcctgacaagggattgagaactttgctgatgtgtatgggagaagcattgagaacaatagcttttaaagttagaacagcttcttgcggtggaacagcttgtgttaattcctttggtgatgaacaactcgctgttgatatgcttgctgataagcttctctttgaggctttgcaatactcgcatgtgtgcaagtatgcttgctctgaagaagtacctgagcttcaagacatgggaggtccagtggaaggtgggtttagtgttgcgtttgatccattggatggatcaagcattgtggatacaaatttcactgtgggaaccatattcggtgtttggcctggagacaagttaaccggaatcactggaggagatcaagtggctgcagccatgggaatctacggtccacgaaccacttatgttttggctgttaagggctttccaggaactcatgagttcttgcttcttgatgaagggaaatggcagcatgtaaaggagacaacagagatcgcagaagggaaaatgttctcaccaggaaacttaagagccacattcgacaactccgaatacagcaagctgattgattactacgtgaaagagaaatacacactgcgatacaccggaggaatggttcctgatgttaaccagattattgtgaaggagaaaggaatcttcacaaatgtgacttctcctacggctaaggcaaagttgaggctgttgtttgaagtggctcctcttggcctgctcatagagaatgctggtggattcagcagtgatggacacaagtccgtgcttgacaagaccatcatcaacctcgacgatagaactcaagttgcttatggctcaaagaacgagatcatccgcttcgaagaaaccctttatggaacatcaagactcaagaatgttcccattggagttaccgcttagagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 25 - DNA, 961 bp, Artificial Sequence
  Sequence design for amiRNA 159 targeting SQE3-1
cctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaagttcagagaaatttgtgaattatacatcatcatcatcatcacggcgcgcctatcttgatctgacgatggaagaagcatgtttcgactatttgacatgagttgagcagggtaaagaaaagctgctaagctatggatcccataagccctaatccttgtaaagtaaaaaaggatttggttatatggattgcatatctcaggagctttaacttgccctttaatggcttttactcttctcaaatagtcgaaacatgcttcatcttctttcaccttctcctcgaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccattaactagtaactaatcccacaaaaatctgagcttaacagcacagttg

SEQ ID NO: 26 - DNA, 961 bp, Artificial Sequence
  Sequence design for amiRNA 159 targeting SQE3-2
cctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaagttcagagaaatttgtgaattatacatcatcatcatcatcacggcgcgcctatcttgatctgacgatggaagcgaaggctggtcaagaacttacatgagttgagcagggtaaagaaaagctgctaagctatggatcccataagccctaatccttgtaaagtaaaaaaggatttggttatatggattgcatatctcaggagctttaacttgccctttaatggcttttactcttctaagttcttgaccagccttcgcatcttctttcaccttctcctcgaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccattaactagtaactaatcccacaaaaatctgagcttaacagcacagttg

SEQ ID NO: 27 - DNA, 961 bp, Artificial Sequence
  Sequence design for amiRNA 159 targeting SQE123-1
cctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaagttcagagaaatttgtgaattatacatcatcatcatcatcacggcgcgcctatcttgatctgacgatggaagcacaatgggcgtttcatacaacatgagttgagcagggtaaagaaaagctgctaagctatggatcccataagccctaatccttgtaaagtaaaaaaggatttggttatatggattgcatatctcaggagctttaacttgccctttaatggcttttactcttcttgtatgaaacgcccattgtgcatcttctttcaccttctcctcgaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccattaactagtaactaatcccacaaaaatctgagcttaacagcacagttg

SEQ ID NO: 28 - DNA, 961 bp, Artificial Sequence
  Sequence design for amiRNA 159 targeting SQE123-2
cctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaagttcagagaaatttgtgaattatacatcatcatcatcatcacggcgcgcctatcttgatctgacgatggaagggtcgatgtacctggtcaaaacatgagttgagcagggtaaagaaaagctgctaagctatggatcccataagccctaatccttgtaaagtaaaaaaggatttggttatatggattgcatatctcaggagctttaacttgccctttaatggcttttactcttcttttgaccaggtacatcgacccatcttctttcaccttctcctcgaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccattaactagtaactaatcccacaaaaatctgagcttaacagcacagttg